(19) United States
(12) Patent Application Publication
(10) Pub. No.: US 2016/0106392 A1
(43) Pub. Date: Apr. 21, 2016

(54) ULTRASONIC ARRAY FOR BONE SONOGRAPHY

(71) Applicant: THE GOVERNING COUNCIL OF THE UNIVERSITY OF TORONTO, Toronto (CA)

(72) Inventors: Amir Manbachi, Toronto (CA); Richard S.C. Cobbold, Toronto (CA)

(21) Appl. No.: 14/893,642

(22) PCT Filed: May 23, 2014
(86) PCT No.: PCT/CA2014/050484

§ 371 Date: Nov. 24, 2015

Related U.S. Application Data
(60) Provisional application No. 61/827,276, filed on May 24, 2013.
(60) Provisional application No. 61/827,284, filed on May 24, 2013.

(51) Int. Cl. A61B 8/00; A61B 17/16; A61B 8/08; A61B 8/12; A61B 17/17

(52) U.S. Cl. CPC A61B 8/4488; A61B 8/12; A61B 17/1703; A61B 8/0875; A61B 8/5207; A61B 17/1671; A61B 8/461; A61B 8/54

(57) ABSTRACT

This invention relates to methods and devices for use in ultrasound imaging. Ultrasonic methods, systems and low-frequency annular transducer array devices for bone image guidance, particularly during spinal fusion surgery and the process of pedicle screw insertion are provided.

### CROSS REFERENCE TO PRIOR APPLICATIONS

This application claims priority under the Paris Convention from U.S. Application No. 61/827,276, filed on May 24, 2013 and U.S. Application No. 61/827,284, filed on May 24, 2013, the entire contents of which are incorporated herein by reference.

### FIELD OF THE INVENTION

The present invention relates to methods, systems and devices for ultrasound imaging. More particularly, the present invention relates to methods, systems and devices for image generation and analysis for use in surgical applications such as orthopedic surgery, including spinal fusion surgery and the process of pedicle screw insertion.

### BACKGROUND OF THE INVENTION

It is estimated that up to 40% of the population may be experiencing back pain. Spinal fusion surgery may be recommended if a subject has one or more of the following conditions: (1) fractured vertebrae, (2) spinal curvature deformities (e.g. scoliosis, or kyphosis), and (3) at least one bulging or herniated intervertebral disk, which might press on exiting spinal nerves. Surgical hardware can be used to fix a corrective structure to the spine, such as, for example, pedicle screws that are attached to small bones in the vertebrae called pedicles. Almost 250,000 spinal fusion surgeries were performed during 2008 in the United States alone (American Academy of Orthopedic Surgeons). Almost 650,000 people a year undergo lumbar spinal fusion for a variety of ailments.

Pedicle screw placement is complicated due to limited visibility of the spine, continuous bleeding in exposed regions, close proximity of the pedicle to vital neural and vascular structures and variability in pedicle morphology. Improperly placed pedicle screws can place surrounding neural and vascular structures at risk, including the spinal cord, nerve roots and aorta. Some studies suggest a high rate of pedicle screw misplacement (20-40%), which leads to neurological deficits (e.g. patient paralysis) in up to 3-5% of cases.

Typically, the screw hole is prepared using a cannulation probe (awl-like boring tool) that is advanced through the vertebral cancellous bone in the middle of the pedicle. To avoid improper placement of the screws, the surgeon relies on tactile feedback to differentiate between “soft” cancellous bone, filled with bone marrow in the middle of the pedicle bone, and tougher cortical bone in the surrounding of the pedicle (FIG. 1). If probe advancement becomes difficult (e.g. probe comes in contact with cortical bone) or too effortless (e.g. probe has perforated cortical bone) the surgeon makes a blind correction to the trajectory of insertion. Alternatively, x-ray fluoroscopy can be used for screw placement. However, this approach exposes patients and staff to harmful ionizing radiation and requires interpretation of two-dimensional images in relation to three-dimensional anatomy, which is visually challenging and results in additional assumptions and risk.

One alternative method for facilitating pedicle screw placement is ultrasound image guidance using a miniature ultrasound probe insertable within the pedicle's guide hole, similar to that used for intravascular imaging (U.S. Pat. No. 3,938,502). The objective of such imaging guidance is to identify and judge the distance from the guide hole and the trabecular/cortical bone interface and to determine whether the proposed insertion trajectory is satisfactory based on the distance.

Indeed, ultrasound has been used in vertebral surgeries since the 1990's (e.g., U.S. Pat. No. 5,167,619 and U.S. Pat. No. 5,976,105). However, ultrasound imaging during spinal surgery was not considered until 2003 when a surgical apparatus with an ultrasonic probe for navigation and placement of implants, such as bone screws, through pedicles was developed (U.S. Pat. No. 6,849,047). The apparatus disclosed in U.S. Pat. No. 6,849,047 was used for cannulation, wherein an intra-cancellous pilot channel was created without breaching the cortical bone.

Ultrasound imaging devices, such as that described in U.S. Pat. No. 6,849,047, require an ultrasound signal to travel from a transducer element, through porous cancellous bone to the tougher cortical bone shell and echo back (propagating the same length inside the cancellous bone) to be received by the transducer element. The time of signal flight translates into distance, which, along with the amplitude of the received signal, can be translated into a line within resultant image as well as the brightness of pixels on that line.

The design in U.S. Pat. No. 6,849,047, for incorporating ultrasound transducers and drill bits, and subsequent designs (e.g., U.S. Pat. No. 8,203,306) provided uni-directional imaging via side-viewing elements, which required manual rotation of the device in order to image one cross-section (slice) of pedicle. Manual rotation can interfere with the pedicle screw placement procedure due to the potential for human error. Further, although the design in U.S. Pat. No. 6,849,047 apparatus provided real-time monitoring for the single cross-section of tissue adjacent to the tip of the drill bit, it would still be possible for the drill bit to touch or even breach a sector of the cortical layer not seen in the uni-directional image. Therefore, this technique requires the user to judge whether screw insertion trajectory is correct based on a single cross-sectional image that is some distance away from the tip of the drilling device.

In the case of spinal fusion surgery, the prophetic idea of a miniaturized ultrasound device insertable into the center of the pedicle in order to image the pedicle from within has been contemplated (U.S. Pat. No. 6,849,047). However, due to many practical challenges, few experimental images have been demonstrated. Some of the remaining challenges include: (1) lack of proper penetration depth for the ultrasound signal within the bone, (2) appropriate signal-to-noise ratio and hence image quality, (3) a solution for imaging bone from within, in three-dimensions, without any changes to the surgical workflow, and (4) the desire for a technology that could estimate the possibility of potential breaches outside the cortical shell for a given wrong insertion trajectory.

Ultrasonic devices invented previously for use in spinal fusion surgery have used a single element transducer (e.g., U.S. Pat. No. 8,203,306). As a result, in order to obtain images of a pedicle cross-section, manual rotation of the device was necessary. Such rotation introduces human factor challenges, at least because rotation must be done while drilling the guide hole within the pedicle. Experimental evidence regarding the practicality of devices such as those of U.S. Pat. No. 6,849,047 is currently limited.

IntraVascular UltraSound (IVUS), a technique wherein an annular ultrasound array is used to generate a cross-sectional vasculature image, is common in cardiovascular diagnostic imaging. However, there is a major difference between IVUS and results in bone sonography. Ultrasound imaging within bone results in high signal attenuation, which increases with higher transmit frequencies. IVUS imaging is based on successful signal transmission through soft tissue at relatively high frequencies (e.g. >20 MHz). However, ultrasound imaging of trabecular bone has a far higher attenuation over the same frequency range, causing the returned signal to be lost in background noise. Consequently, much lower frequencies (e.g. a few MHz) must be used to image bone using ultrasound signals, which leads to considerable loss in image resolution.

As mentioned above, one drawback to devices such as those disclosed in U.S. Pat. No. 6,840,047 is the need for manual rotation of the device. Cylindrically organized phased array transducers can eliminate the need for manual rotation of the transducer when examining hollow organs such as the heart or vasculature prone to plaque buildup. One of the first array transducers had 32 rectangular elements on the circumference of the cylinder to form various imaging lines (U.S. Pat. No. 3,938,502). At any given point in time, 8 elements were employed to acquire a real-time image in each angular direction. Each element was employed both as a transmitter and a receiver. However, such rotational arrays were designed for high frequencies (e.g., >20 MHz) that are not useful for imagery in hard tissue, such as bone.

Achieving adequate signal penetration depth in bone is challenging, at least due to bone's high impedance and reflection characteristics. As a result, bone sonography typically requires the use of low-frequency imaging (e.g. f₀˜1-2 MHz). The use of lower frequencies translates into the application of larger transducer elements because the thickness of each element must be at least half the wavelength associated with the center frequency (λ/2=c/2f). Larger transducer elements are not practical for use with pedicle surgery due to the small size of the target bone.

In some techniques, due to the high frequency of the intravascular ultrasound probe catheter, ultrasound beam reflection from the inner wall of the trabecular bone was near total. This prevented the ultrasound beam from penetrating any significant distance into the trabecular bone, prohibiting imaging of the cortical wall.

Cancellous (trabecular) bone has a very complex structure consisting of a matrix of connected plates and rods, called trabeculae (FIG. 1). These spongy structures are interspersed with marrow. The trabeculae are not arranged uniformly, but tend to align in accordance with the stress distribution in the bone. This inhomogeneous, anisotropic composition makes it very difficult to predict and interpret the propagation of acoustic waves in bones.

It is an object of the present invention to provide an ultrasonic device for bone sonography to obviate or mitigate at least some of the above-mentioned disadvantages.

### SUMMARY OF THE INVENTION

The present invention relates to an apparatus and methods of imaging bone with ultrasound.

In a first aspect of the present invention, an annular ultrasound transducer array is provided. The annular ultrasound transducer array comprises a plurality of transducer elements arranged in a ring configuration, wherein the plurality of transducer elements comprises elements configured to transmit ultrasound signals and elements configured to received ultrasound echoes, and wherein the ultrasound signal is transmitted at a frequency in a range of 0.5 to 5 MHz.

In some embodiments of the present invention, the annular ultrasound transducer array further comprises a plurality of the ring configurations arranged adjacent to one another in a cylindrical configuration, each ring forming a row in the cylindrical configuration.

In some embodiments of the present invention, the transducer array is phased.

In some embodiments of the present invention, the transducer elements in every other row are transmitters and the transducer elements in the rows between the transmitters are receivers. In preferred embodiments of the present invention, the diameter of the ring configuration is in a range of 3 to 5 mm.

In some embodiments of the present invention, the transducer array is configured to be mounted on or in a tool for probing or cannulating bone. In some embodiments of the present invention, the transducer array is integrated with a tool for probing or cannulating bone. In preferred embodiments of the present invention, the tool is for generating pedicle guide holes or pedicle screw placement.

In some embodiments of the present invention, the ultrasound signal to be transmitted is processed by coded excitation and wherein the received ultrasound echoes are processed by de-coding of coded excitation.

In some embodiments of the present invention, the plurality of transducer elements are configured to transmit ultrasound signals in a non-simultaneous, sequential manner.

In some embodiments of the present invention, the transmitted ultrasound signals are directionally focused at an angle less than 90 degrees relative to the longitudinal axis of the cylindrical configuration.

In some embodiments of the present invention, the transducer is in communication with an imaging processor.

In some embodiments of the present invention, the image is generated in real time as the transducer is transmitting ultrasound signals and receiving ultrasound echoes.

In a second aspect of the present invention, a method for producing an image using an ultrasound system is provided. In some embodiments, the method comprises: a) acquiring ultrasound data by: i) transmitting a plurality of ultrasound signals directed outwardly at a bone to be imaged, wherein the signals are transmitted at frequencies in the range of 0.5 to 5 MHz, wherein the signals are reflected by features within the imaged object to produce echoes; ii) measuring the echoes, wherein the measured echoes include echoes reflected from multiple spatial locations within the bone to be imaged; and b) producing an image of the bone from the received echoes.

In some embodiments of the present invention, the outwardly directed ultrasound signals are transmitted by a plurality of transducer elements arranged in a ring configuration, wherein the echoes are received by the plurality of transducer elements, wherein the plurality of transducer elements are in communication with an imaging processor, and wherein the image produced is a cross-sectional image.

In some embodiments of the present invention, the outwardly directed ultrasound signals are transmitted by a plurality of transducer elements arranged in a first plurality of ring configurations, wherein the echoes are received by a second plurality of ring configurations, wherein the first and second plurality of ring configurations are arranged adjacent to one another in a cylindrical configuration, each ring forming a row in the cylindrical configuration and wherein the image produced is a cylindrical or conical image.

In some embodiments of the present invention, the plurality of adjacent rings are mounted to or in or integrated with a tool and wherein the tool is inserted in the object to be imaged.

In some embodiments of the present invention, the method further comprises ultrasound signals directed forwardly relative to the insertional trajectory of the tool, wherein the forwardly directed ultrasound signals are transmitted from a plurality of the transducer elements and wherein the image produced is a conical image, wherein the apex of the cone is ahead of the tool along the insertional axis.

In some preferred embodiments of the present invention, the imaged bone is a pedicle bone. In some preferred embodiments of the present invention, the image is generated in real time.

In a third aspect of the present invention, a method for predicting pedicle cortical breach is provided. In some embodiments, the method comprises: a) inserting into the pedicle a tool comprising an annular ultrasound transducer; b) acquiring ultrasound data by: i) transmitting from the annular ultrasound transducer a plurality of ultrasound signals directed both outwardly and forwardly relative to the insertional trajectory of the tool, wherein the signals are transmitted at a frequency in a range of 0.5 to 5 MHz, wherein the signals are reflected by features within the pedicle to produce echoes; ii) measuring the echoes using a the annular ultrasound transducer, wherein the measured echoes include echoes reflected from multiple spatial locations within the pedicle; c) producing an image of the pedicle from the received echoes, wherein the image includes the cortical boundary of the pedicle, wherein a spatial relationship between the inserted tool and the cortical boundary is depicted in the image; and d) predicting the possibility for cortical breach based on the image obtained in step c).

In some embodiments of the third aspect of present invention, the tool is a cannulation probe or drill.

In some embodiments of the third aspect of present invention, the ultrasound signals to be transmitted are processed by coded excitation and wherein the echoes are processed by de-coding.

In a fourth aspect of the present invention, a system for producing an image of bone using an ultrasound system is provided. In some embodiments, the system comprises: a) a phased annular transducer comprising a plurality of transducer elements arranged in a ring configuration, wherein the plurality of transducer elements comprises elements configured to transmit ultrasound signals and elements configured to received ultrasound echoes, and wherein the ultrasound signal is transmitted at a frequency in a range of 0.5 to 5 MHz; b) a tool configured to probe or cannulate bone, wherein the tool comprises the phased annular transducer; c) an imaging processor in communication with the phased annular transducer; d) an imaging display coupled with the imaging processor; and e) an electronic controller coupled with the tool and the phased annular transducer, wherein the electronic controller is configured to control the operation of the tool to move the tool in a desired direction.

In some embodiments of the fourth aspect of the present invention, the phased annular transducer further comprises a plurality of the ring configurations, wherein the plurality of ring configurations are arranged adjacent to one another in a cylindrical configuration, each ring forming a row in the cylindrical configuration.

### BRIEF DESCRIPTION OF THE DRAWINGS

The features of the invention will become more apparent in the following detailed description in which reference is made to the appended drawings wherein:

FIG. 1 depicts the anatomy of soft cancellous bone and hard cortical bone encapsulating the cancellous bone.

FIG. 2 is a design schematic of an annular ultrasound transducer employed for obtaining cross-sectional images, in accordance with one embodiment.

FIG. 2a is a pictorial representation of an annular ultrasound transducer, wherein the transducer involves two layers of acoustic matching, in accordance with one embodiment.

FIG. 3 is a block diagram illustrating components of the transducer array for communicating with a computing device, in accordance with one embodiment for transmitting ultrasound waves and measuring received echoes from a subject.

FIG. 4 depicts a real-time cross-sectional ultrasound image of pedicle bone generated using an annular ultrasound transducer array of the present invention, in accordance with one embodiment.

FIG. 5 is a pictorial representation showing electronic focusing of ultrasound beams in pedicle cross-sectional imaging, in accordance with one embodiment.

FIG. 6 is a pictorial representation of an exemplary sound pressure profile simulation comparing the use of rotational phased arrays (as opposed to single element transducers), in accordance with one embodiment.

FIG. 7 depicts an example of an annular ultrasound transducer having a plurality of ring-shaped transducer arrays, in accordance with one embodiment.

FIG. 8 is a perspective diagram providing exemplary design specifications of an angular sector arch of the cylindrical ultrasound transducer array (an example of which is shown in FIG. 7), in accordance with one embodiment.

FIG. 9 is a pictorial representation of a cylindrical ultrasound transducer array incorporated with a drill bit for simultaneous imaging and pilot hole creation in a pedicle bone structure.

FIG. 9A is a diagram of a 32-element ultrasound transducer probe, handle and array (embedded within an epoxy protective layer), in accordance with one embodiment.

FIG. 10 is a pictorial representation of electronic steering of ultrasound beams, wherein the beams are directed forwardly relative to the insertional direction of the transducer array (e.g., the array of FIG. 7 or 8), in accordance with one embodiment.

FIG. 11 is a schematic that illustrates how the ultrasound beam focal spot of the ultrasound transducer array is shifted spatially using a phased array technique of introducing electronic delays to the fire timing of each element, thereby allowing the user of the array to “look ahead” of the array (e.g., the array of FIG. 7 or 8), in accordance with one embodiment.

FIG. 12 is a block diagram illustrating exemplary functional components involved in ultrasonic imaging of bone using the method of the present invention, in accordance with one embodiment.

FIG. 13 is a block diagram illustrating exemplary steps for measuring the radial distance between a surgical tool and the cortical boundary of a pedicle, wherein the user is alerted if the distance is indicative of a potential cortical breach, in accordance with one embodiment.

FIG. 14 is a block diagram further illustrating the exemplary steps of a method for predicting cortical breach according to present invention, in accordance with one embodiment.

FIG. 15 is a block diagram further illustrating the method for predicting cortical breach according to the present invention, in accordance with one embodiment.

FIG. 16 is a block diagram further illustrating the method for predicting cortical breach according to the present invention, in accordance with one embodiment.

FIG. 17 is a chart depicting exemplary results of design models revealing an electrical resistance of around 50Ω for each ultrasound element around the ring sensor array (after electronic matching).

FIG. 18 is a chart depicting −8 dB (˜40% ratio) energy loss at the interface of electronics with the acoustics hardware (ideal transformer of 1:16 ratio is applied).

FIG. 19 are exemplary views depicting a sample of a Graphical User Interface used for designing a desirable low-frequency rotational phased array, in accordance with one embodiment.

FIG. 20 depicts multiple B-mode (ultrasonic Brightness Mode) images along the length of a simple cylindrical hollow structure, mimicking the circumstances under which the prediction of potential cortical breach should function. The cylinder is shown as a simplified example of the anatomical structure of the pedicle bone.

FIG. 21 depicts the insertion trajectory of a pedicle probe, various cross-sectional images of the pedicle and the corresponding pedicle bone cross section.

FIG. 22 graphically depicts an exemplary simplistic cannulation trajectory through a pedicle (square) relative to the dorsal (diamond) and vertical (triangle) cortical layers.

FIG. 23 is a chart depicting properties of exemplary fabricated materials for the transducer arrays for bone sonography, in accordance with one embodiment.

FIG. 24-28 illustrate exemplary transducer captured images in accordance with experimental testing, based on a custom hardware designed and fabricated for bone sonography and a custom software that drives the transducer array.

FIG. 29 illustrates one embodiment of the transducer array device and an exemplary acoustic pressure profiles for same, when using one 45° element sector at a time to image.

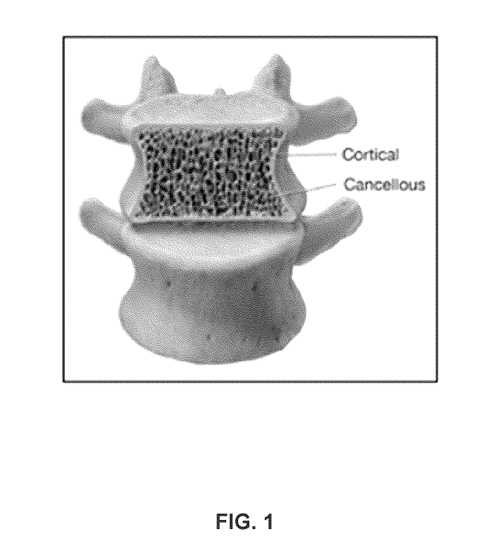

### DETAILED DESCRIPTION OF THE INVENTION

Unless defined otherwise, all technical and scientific terms used herein generally have the same meaning as commonly understood by one of ordinary skill in the art to which this invention belongs. For convenience, like numerals in the description refer to like structures in the drawings.

As will be understood by a person skilled in the art, the dimensions illustrated in the accompanying figures are exemplary but not limiting. Other variations may be envisaged.

The present invention generally relates to methods and devices for imaging bone using ultrasound energy. To achieve an adequate ultrasound signal penetration depth in bone, it is necessary to use low-frequency signals. Preferably, the device of the present invention transmits an ultrasound at a low frequency in a range of 0.5 to 5 MHz.

Known low frequency techniques require use of a relatively large transducer that would not meet the constraint of fitting within a pedicle's bore hole. Known devices that can fit within the pedicle bore hole have a single ultrasound transducer that transmits and receives a signal in one direction only. Therefore, to obtain a circumferential image of the pedicle bore hole, the user must manually turn the device to obtain multiple images that can be put together to obtain a circumferential image. In contrast, in some aspects of the present invention, a low frequency annular transducer array that fits within a pedicle bore hole is provided and may be used to image a pedicle.

In some embodiments, the annular ultrasound transducer comprises a plurality of transducer elements arranged in a ring configuration. Transducer elements are configured to transmit ultrasound signals and/or receive ultrasound echoes.

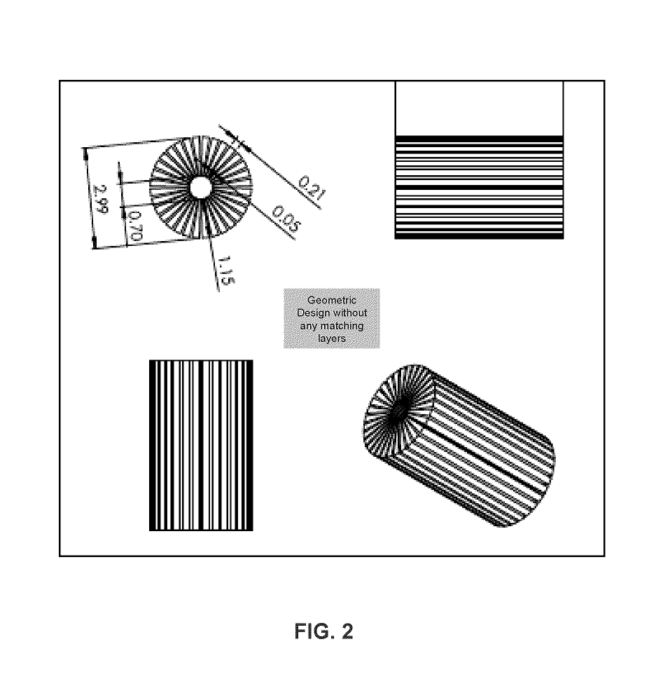

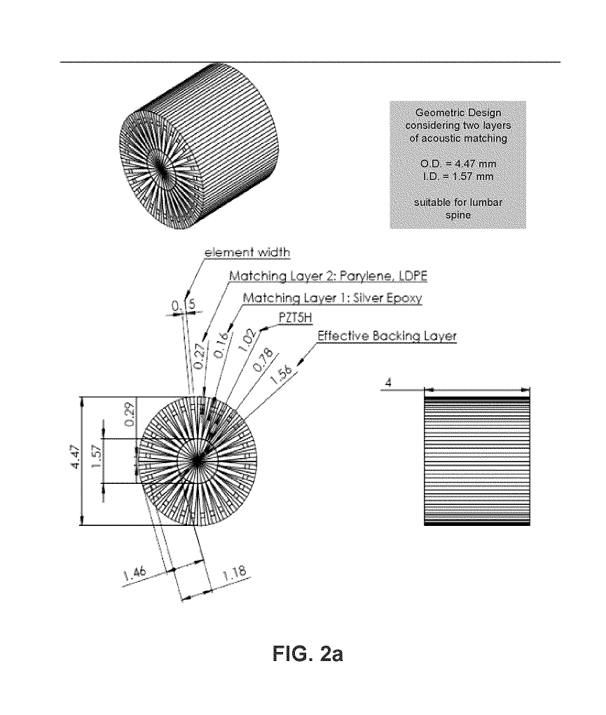

Referring to FIG. 2, in some embodiments, the annular transducer of the present invention can be designed without matching layers. An acoustic matching layer is made from a material that has acoustical impedance (sound resistance) that is between the active transducer element and the imaging media. A matching layer is provided to minimize the energy loss experienced by the ultrasound wave as it travels from one media to another. In some embodiments, the absence of a matching layer permits the device to fit more easily into a pedicle's bore hole. However, due to acoustic impedance mismatch between the piezoelectric transducer and the human tissue, a larger energy loss will occur at the interface of the layers, which will decrease sensitivity of an image obtained using such a device. Referring to FIG. 2, shown are various aspect views of an exemplary annular ultrasound transducer where ultrasound signals are transmitted and echoes are received by the transducer. The dimensions shown are exemplary only.

Referring to FIG. 2A, in some embodiments, the annular transducer of the present invention can be designed to consider up to two layers of acoustic matching. Inclusion of additional layers might involve a tradeoff between preference for a smaller device and improved ultrasound image quality. In some embodiments, the diameter of the cylindrical transducer array of the present invention is in a range of 3-5 mm, depending on the anatomical region, patient-specific demands and user preference. Other diameters can be used depending upon the use of the annular transducer array and the desired ultrasound image quality.

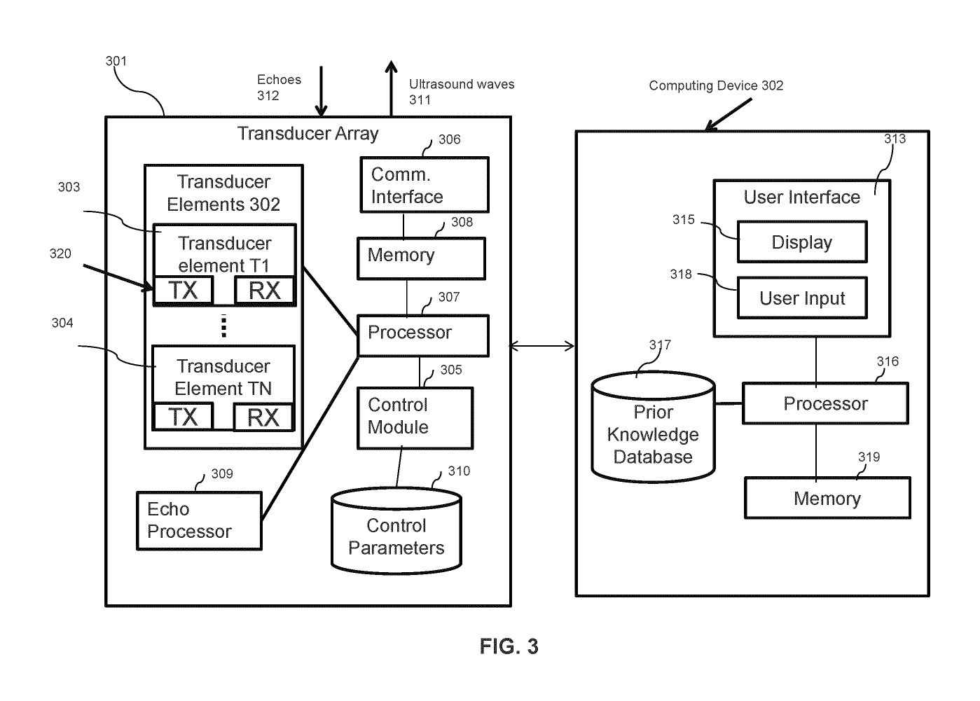

Referring to FIG. 3, the transducer comprises a transducer array 301. The transducer is annular, cylindrical, or conical in shape depending upon the type of subject (e.g. pedicle bone) being imaged and the desired focus/signal to noise ratio. As described herein, the annular transducer can be configured with or without matching layers. Whether matching layers are used is defined by the size of the transducer device allowed for the pedicle bone (e.g. absence of matching layer allows smaller device), or increased desired sensitivity (e.g. increased sensitivity is provided by matching layers). The parameters for configuring the transducer can be stored in a database (e.g. prior knowledge database 317) on the computing device 302. In this manner, the efficiency of a particular use and images obtained from a transducer are stored in database 317 for defining matching parameters by the processor 316 for subsequent transducer use.

Referring to FIG. 3, the transducer array 301 comprises one or more transducer elements (303, 304). Each transducer element 303, 304 further comprises a transmitter (TX) and/or a receiver (RX) elements 320. The transmitter elements are configured for transmitting the ultrasound waves 311 to a subject (e.g. a pedicle bone) and the receiver elements are configured for receiving the echoes 312. The operation of the transducer elements 302 is controlled by a processor 307 in communication with a control module 305 for triggering the operation of one or more transducer elements 303 in generating the transmitter and/or receiver signals. As will be described, the control module 305 is further in communication with control parameters 310 for defining timing, delay and selection of one or more transducer elements 302. In some embodiments, the transducer array 301 is in communication (e.g. via a communication interface 306) with an external computing device 302 for generating the images from the received echoes 312. The transducer array 301 may be directly electrically coupled to the computing device 302 or may be in wireless communication therewith (e.g. Bluetooth). Referring to FIG. 3, the annular transducer array 301 is in communication with an imaging processor 316 and an image display 315 for generating images of the pedicle bone.

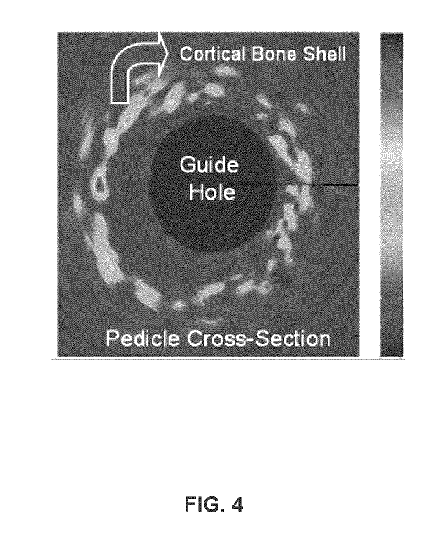

In one embodiment, in response to generating the ultrasound waves 311 to a subject (e.g. a pedicle bone), the subject tissue provides one or more echo signals 312. The transducer array 301 is configured to receive the echo signals 312 and process the echo signals 312 via an echo processor 309. The echo processor 309 is configured to translate the echo signals 312 (e.g. by averaging, defining a specific focal point to provide emphasis to particular echo signals, by ranking the echo signals and providing a weighted gain) to a response signal indicative of the image of the structure. The response signal is provided to the computing device 302 for processing by the processor 316 and generating the image on the display 315. Referring to FIG. 3, the computing device 302 further comprises a user interface 313 for receiving user input 318 to manipulate the image and/or provide control parameters for affecting the resolution, timing, and/or delay as stored in the control parameters 310. In this manner, cross-sectional images of a hollow structure can be obtained by ultrasound ‘pulse-echo’, which is based on the time that it takes for an excitation pulse to travel within the bone, hit the thick cortical target and return back to the transducer. For example, a cross-sectional image of a pedicle is shown in FIG. 4.

Referring to FIG. 3, the computing device 302 further comprises a memory 319 for storing instructions thereon and for execution by the processor 316 in generating the image from the echo signals 312 as provided by the echo processor 309.

Referring to FIG. 3, in some embodiments, the plurality of transducer elements 302 are arranged in a ring-like configuration that allows a circumferential image to be taken in real time (e.g. as provided to an image processor 316 for generating the image on a display 315). The transducer elements 302 are configured to transmit ultrasound signals 311 and/or receive ultrasound echoes 312 for generating the image.

Referring again to FIG. 3, one advantage of a transducer array 301 having a plurality of transducer elements 302 is that a user can deliberately fire the elements one at a time (e.g. trigger the operation of one or more transmitter 320 in one or more transmitter elements 302), in sequence, simultaneously (e.g. multiple transmitter elements 303 and 304) or with delays with respect to adjacent elements.

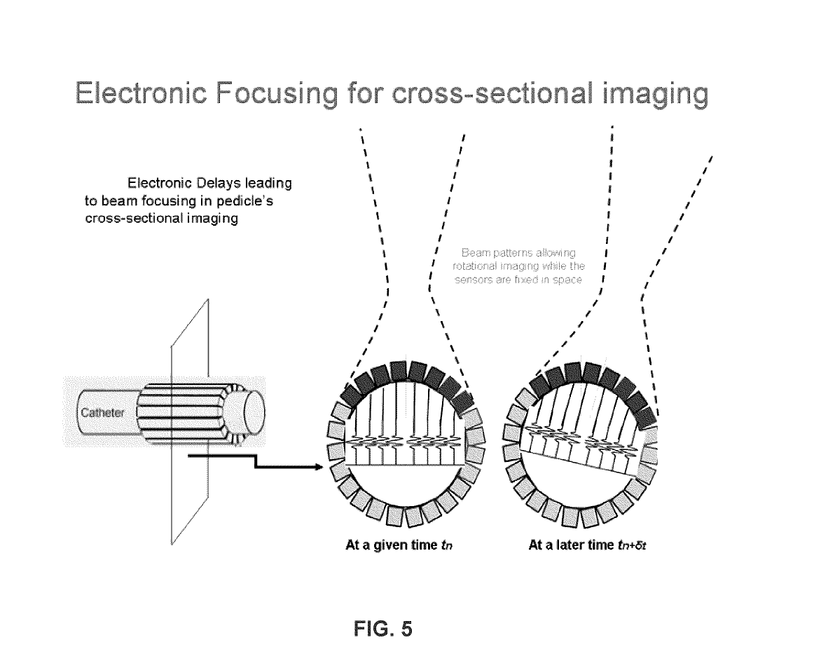

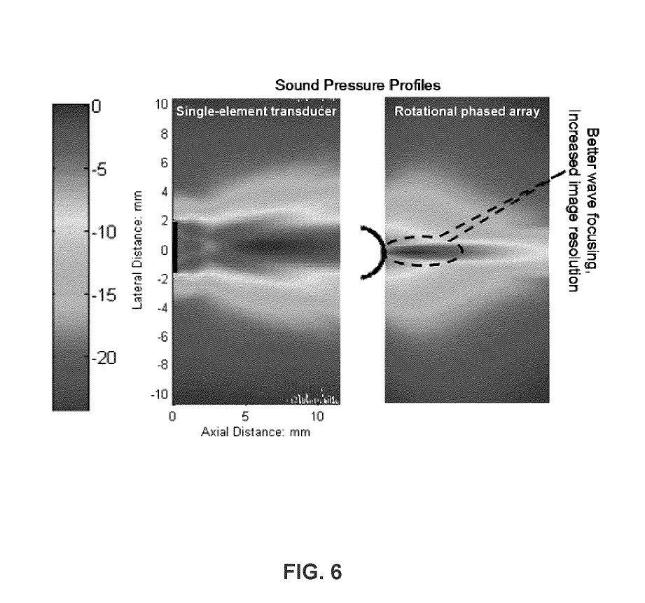

Indeed, in some embodiments of the present invention, the transducer array 301 can transmit ultrasound signals 311 from a specified sub-set of transducer elements 302. The sequence of transmitted ultrasound signals 311 could include desired time delays, which might be useful for improving focus of the ultrasound waves (i.e., beams), which could result in improved image resolution and quality. For example, when waves originating from two or more sources interact with one another, phasing effects occur, which lead to an increase or decrease in wave energy at the point of wave combination. When elastic waves of the same frequency meet such that their displacements are synchronized (i.e., in phase or at 0 degree phase angle), the wave energies combine and create a larger amplitude wave. If they meet such that their displacements are opposite (i.e., 180 degrees out of phase), then the wave energies will cancel each other. When elastic waves meet at phase angles between 0 and 180 degrees, a range of intermediate stages between full addition of energy and full cancellation of energy can occur. By varying the timing of the waves from a large number of sources, it is possible to use these effects to both steer and focus the resulting combined wave front as shown, for example, in FIGS. 5 and 6. The timing information of the waves for triggering the operation of one or more transducer elements 302 is stored in the control parameters 310 for use by the control module 305 in affecting the selection, phase, timing and triggering the operation of the transducer elements 302. Referring to FIG. 3, one or more instructions may be stored on a memory 308 for affecting the operation of the transducer elements 302 in generating the ultrasound waves 311 and/or analyzing the echo signals 312.

In some embodiments, the annular ultrasound transducer arrays of the present invention are phased (e.g. as stored in the control parameters 310 for use by the control module 305), increasing speed and ease of use of the transducer array. Further, unlike a single transducer element probe, manual rotation is not required to generate a cross-sectional image when using the annular transducer of the present invention.

Phased array systems pulse and receive signals from the plurality of elements of an array. The plurality of elements 302 is pulsed in a pattern to cause multiple beam components to combine with each other to form a single wave front 311 traveling in the desired direction. Similarly, the plurality of receiver elements 320 combine the echo input 312 into a single presentation. Because phasing technology permits electronic beam shaping and steering, it is possible to generate various ultrasonic beam profiles from a single probe assembly.

It is contemplated that in some embodiments (e.g. FIG. 3), instructions stored in the memory 308 can be used by the control module 305 for execution by the processor 307 to control ultrasound beam angle, focal distance, and beam spot size (e.g. control parameters 310). These parameters can be dynamically scanned at each inspection point to optimize incident angle and signal-to-noise for each part geometry.

It is contemplated that in some embodiments, multiple-angle inspection can be performed with a single, small, multi-element probe and wedge, offering either single fixed angles or a scan through a range of angles. This method provides greater flexibility for inspection of complex geometries, such as cancellous bone. It is also contemplated that in some embodiments, multiplexing across many elements could allow motionless high-speed scans from a single transducer position. More than one scan may be performed from a single location with various inspection angles.

In some embodiments, as shown in FIGS. 2A, 3 and 7, the annular ultrasound transducer 301 comprising a plurality of transducer elements 302 arranged in a ring configuration (i.e., the single ring transducer array) fires a number of elements at a time (e.g., 8 elements are fired at one time). The device then receives echoes 312 in the same elements 302 (i.e., elements are both transmitters and receivers). Upon completion, in one example, the whole process takes about 10-20 psecs, the echoes 312 are electronically processed and stored (e.g. via the echo processor 309). Subsequently, in one example, the next set of (8) elements are selected (for example, instead of elements 1 to 8, elements 2 to 9 are selected) as defined by the control module 305 and fired, and the process continues iteratively until the full circumference of the array 301 has fired and received signals and echoes 312. Thus, in some embodiments, the annular ultrasound transducer array 301 can be used to generate a cross-sectional imaging of a pedicle bone without requiring rotation or movement of the device. In preferred embodiments, a cross-sectional image will be formed by 32 focal spots dispersed 360° around the annular transducer at an angular distance of 360°/32=11.25° from one focal spot to the next.

In some embodiments, 32 transducer elements with an aperture size of 4-8 elements (total number of active firing elements at a given time) provide a balance between image quality, practicality and cost-effectiveness of device fabrication.

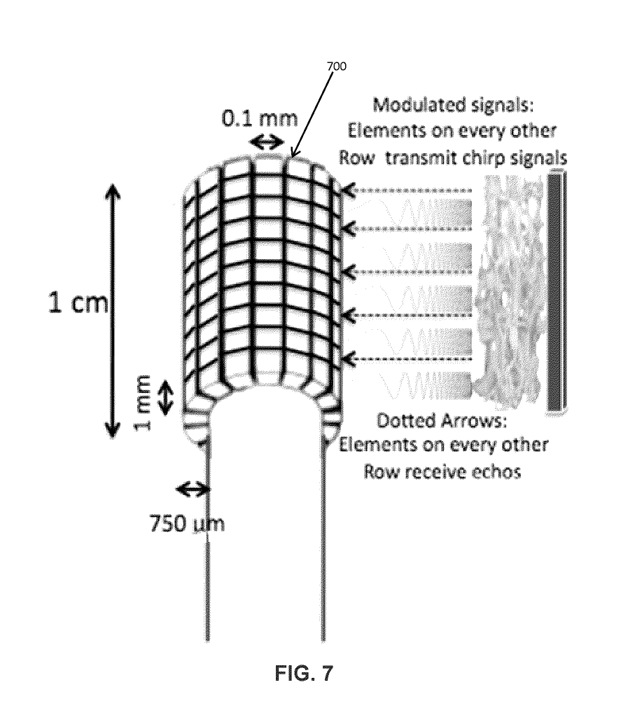

In some aspects of the present invention, the annular ultrasound transducer comprises a plurality of the ring configurations 700 (e.g. shown in FIG. 7). The cylindrical ultrasound transducer array illustrated in FIG. 7 is configured for generating a three-dimensional image of the pedicle bone's cortical layer. Referring to FIG. 7, the dimensions are provided for exemplary purposes, providing proof of principle, particularly for fitting the transducer into the pedicles of lumbar spine. Dimensions can be varied to suit pedicle morphology differences in lumbar, cervical or thoracic pedicle bones. In some embodiments, the plurality of ring configurations 700 are arranged adjacent to one another in a cylindrical configuration, each ring forming a row in the cylindrical configuration. Such a configuration allows for multiple cross-sectional images to be taken concurrently, which can then be configured to form a three dimensional image of the bone structure surrounding the transducer array.

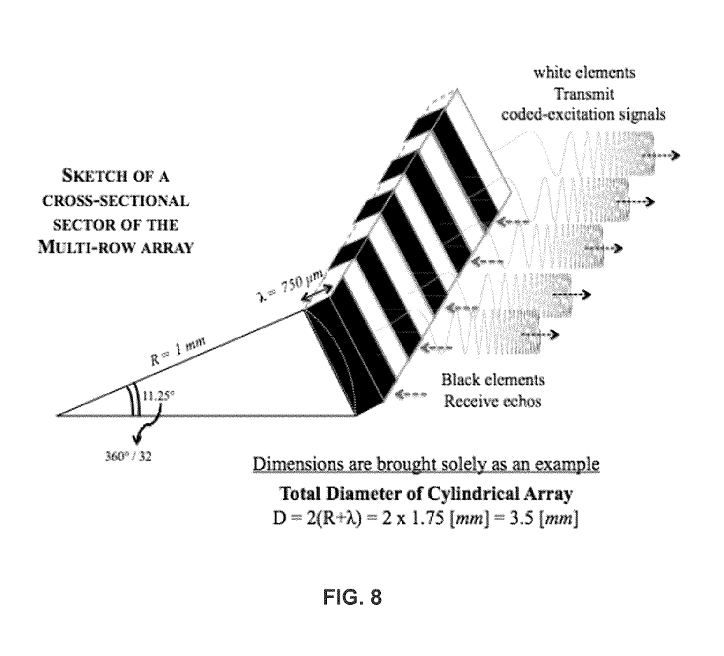

In some embodiments, the transducer elements in every other row of the plurality of rings are transmitters and the transducer elements in the rows between the transmitters are receivers (FIG. 7 and FIG. 8). This design is particularly useful when the ultrasound signal is Chirp Modulated, at least because chirp modulated ultrasound signals comprise transmitted pulses that are longer in length than un-modulated signals. Longer pulses allow for a possibility of overlap between transmitted and received signals. As a result, in some embodiments, the transducer array imaging system could employ this alternative row design to overcome potential signal overlap.

Referring to FIG. 8, shown is a schematic of a linear array that sits at a specific angle sector. Putting together a number of these arcs will generate a multi-ring or multi-row array of a cylindrical transducer. The dimensions shown in FIG. 8 are exemplary and not limiting. Referring to FIG. 8, each sector includes a single element at a circular cross-section and there are 32 elements across the circumference, 8 of which are to be employed simultaneously to obtain focused angular images. The dimensions are shown as a proof of principle, particularly for fitting into an example subject such as the pedicles of lumbar spine. Accordingly, the dimensions vary based on variability of pedicle morphology from lumbar to cervical (neck) or thoracic (chest) spine.

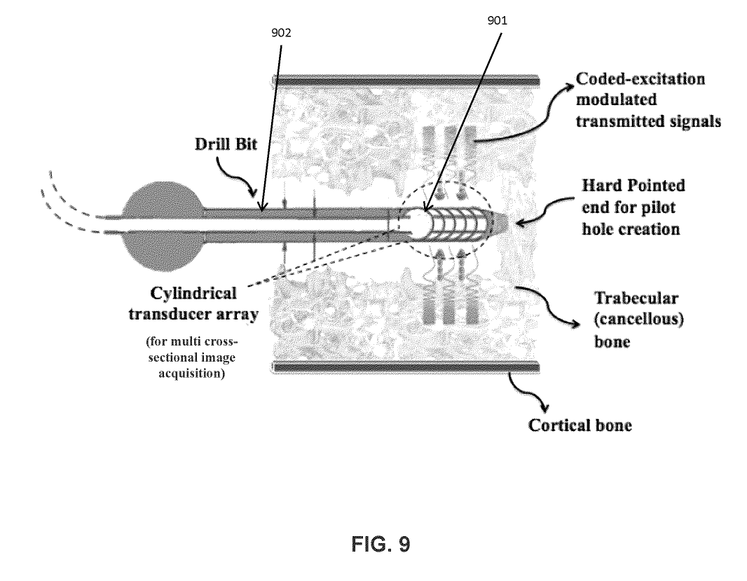

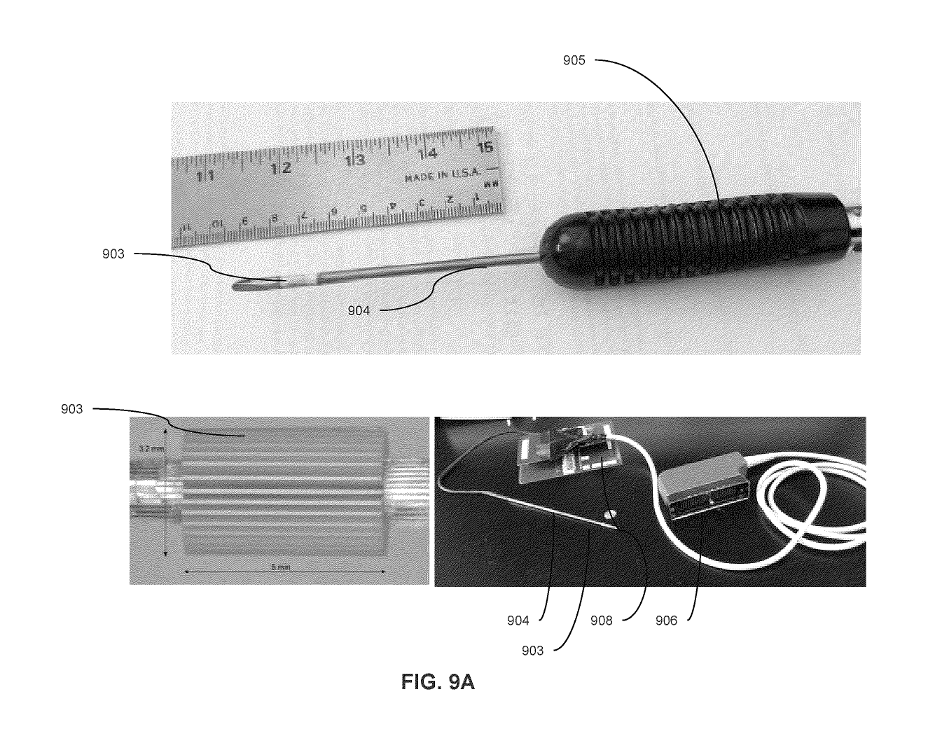

In some embodiments of the present invention, the annular ultrasound transducer array 901 is configured to be mounted on or in a tool 902 (e.g. a drill bit) for probing or cannulating bone (FIG. 9). The device 900 integral with the array 901 is shown inserted within the anatomical structure of a target subject.

In other embodiments, the transducer array is integrated with a tool for probing or cannulating bone (e.g. as shown in FIG. 9A). For example, the transducer array of the present invention can be mounted to or integrated with a tool for generating pedicle guide holes or a tool used for pedicle screw placement.

Referring to FIG. 9A, shown is an ultrasound transducer array probe and handle in accordance with one embodiment. The device comprises a surgical toolkit resembling screwdriver geometry (e.g. tool bit 904), a transducer array embedded inside an epoxy (to protect it from scratches from bone) 903 and a handle portion 905. The tool bit 904 is for engaging with a treatment surface (e.g. bone tissue) and for penetration of same. The transducer array 903 is configured for providing radial imaging from within the target, preferably with a low frequency transducer as described herein to allow for penetration of the tissue while considering signal to noise ratio of the captured image, and preferably having relatively small dimensions. FIG. 9A provides a 32 element ultrasound transducer probe configured for imaging the pedicle bone radially, from within, without mechanical rotation of the element (e.g. transducer array 903). In one aspect, the transducer array 903 is driven using electronic steering rotation in order to obtain cross sectional images (e.g. 360 degree radial imaging) from the pedicle bone. Referring to the bottom right image in FIG. 9A, shown is the transducer array 903 mounted on a stainless steel rod 904 connected to an electrical matching circuitry 908 that reduces the signal loss as the signal travels through the electronic components. An electrical connector socket 906 is used to interface the hardware to an ultrasound console, whereby the custom-developed software is employed (as described on FIG. 3) for facilitating the capturing of images and communicating the reflected echo information (e.g. one or more of control parameters 310 shown in FIG. 3, such as but not limited to: control of timing, delays, direction, electronic focusing, and number of transducer elements engaged at one time for sending the ultrasound waves)

It is contemplated that in some embodiments the transducer array 903 is driven by a motor (e.g. a stepper motor with radial rotation) rather than, or in addition to, electronic steering.

Referring again to FIG. 9A, the transducer array 903 is preferably a 32 element ultrasound imaging array, operating in a low frequency range. The transducer array 903 consists of 32 transducer elements disposed on a cylindrical configuration and embedded within a coating such as an epoxy that protects the elements from scratches from interaction with the bone. In one aspect, the array is configured for being coupled with a tool bit (e.g. a screwdriver tip).

It is contemplated herein that devices of the present invention could be sterilized and reused in surgery, at least for example, by using low-temperature sterilization methods, for example, those involving hydrogen peroxide or ethylene oxide gas.

A skilled artisan will appreciate that various embodiments of the present invention are advantageous relative to x-ray technologies. For example, ultrasound technology offers portability, increased safety and decreased cost, relative to x-ray and ultrasound imaging can provide real time feedback to a surgical team without causing deviation from routine surgical workflow.

It is contemplated herein, that in some embodiments, the speed of sound in the media in which the wave is propagating can be adjusted depending upon the osteoporosity of the target bone. For example, the user has the option of investigating the resolution of the design parameters when the wave is travelling purely in bone, purely in blood, or even in a mixed media with ratios such as 30%-70%, 50%-50%, etc. In some embodiments, the user could select incorporation of attenuation in the results.

It is contemplated herein that annular ultrasound transducers of embodiments of the present invention can transmit excitation-coded ultrasound signals and that echoes of coded signals can be decoded after receipt in the transducer element(s). It is contemplated that methods of signal excitation coding, such as chirp modulation, golay coding and those described in U.S. Provisional patent application titled “Ultrasonic Signal Processing for Bone Sonography”, filed May 24, 2013, which names the inventors of the present application as inventors, can be used with embodiments of the transducer array and/or method.

Some aspects of the present invention involve a method for producing a cross-sectional image of bone using an ultrasound system. The method comprises acquiring ultrasound data by: i) transmitting a plurality of low frequency ultrasound signals directed radially at a bone to be imaged, wherein the signals are reflected by features within the bone to produce echoes; ii) measuring the echoes, wherein the measured echoes include echoes reflected from multiple spatial locations within the bone; and producing an image of the bone from the received echoes.

In some embodiments, the method for producing an image of bone using ultrasound further comprises noise reduction by signal (image) averaging. In this method, a plurality of ultrasound signals (modulated or un-modulated) is transmitted at the bone to be imaged (e.g. waves 311 shown in FIG. 3). The echoes of these signals 312 are received and averaged (e.g. via echo processor 309). Averaging reduces the random noise relative to the signal. Transmitting a plurality of ultrasound pulses and averaging the received echoes 312 of the pulses allows generation of a plurality of images from which an average can be taken, which minimizes the effect of random noise. The process of averaging multiple images is preferable when the target is invariant, such as bone.

In some aspects, the image produced using embodiments of the method can be a real time image. As used herein, a real time image is generated in a range of micro- to milliseconds. For example, if 32 rotations of the annular transducer array are required, and an image is generated every 10 psecs, 32 images would be generated in 320 psecs. If time delays for electronic transmission and data processing are added to this time, an image generated in less than a second is feasible. Such an image is referred to herein as a real time image.

In some embodiments, the annular ultrasound transducer device having a single ring configuration, as disclosed herein, can be used to transmit and receive ultrasound signals 311 and echoes 312 in the method. Such a device can be configured to communicate with an imaging processor (e.g. processor 316 in FIG. 3) to produce a cross-sectional image of the bone, using the method of the present invention (e.g. as shown in FIG. 3).

In some embodiments, the annular ultrasound transducer device having a plurality of ring configurations, as disclosed herein, can be used to provided the present invention. Such a device can be configured to communicate with an imaging processor (e.g. processor 316 in FIG. 3) to produce a three-dimensional cylindrical image of the bone.

In some embodiments, the method involves using an annular ultrasound transducer that is mounted to or in or integrated with a tool that can be inserted into the bone to be imaged (e.g. as shown in FIGS. 9 and 9A).

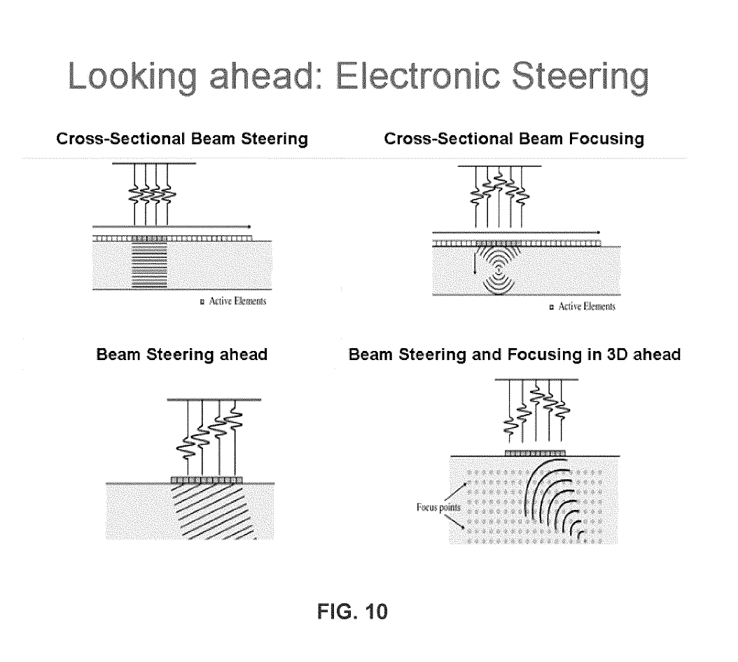

In some embodiments, the method involves directing forwardly ultrasound signals from at least one of the transducer elements relative to the insertional trajectory of the tool, such that the image produced from the received echoes is conical image, wherein the base of the cone is ahead of the tool along the insertional axis. Generation of a real time image that includes both cross-sectional and forwardly directed spatial information is advantageous, at least because it provides the user with information regarding the present location of the transducer array relative to the three-dimensional anatomy of the bone being imaged. (FIG. 10)

In some embodiments, beams are focused to look ahead at an angle relative to the axis of tool insertion. When electronic delays are introduced to each transducer element in the annular transducer, the device can conduct linear sweeping in order to focus the sound waves at an angle relative to the axis of insertion (for example 30 degrees) and hence can “look ahead.” (FIG. 10)

Referring to FIG. 11, phased array techniques, such as introducing electronic delays to the timing that each transducer element fires, can be used to shift the focal spot of the ultrasound signals (e.g. as stored within the control parameters 310 for use by the control module 305 in controlling the operation, selection triggering, timing and/or phase of one or more transducer elements 302). This allows the user to look ahead in the targeted pedicle, to see cross-sections of bone that have not yet been reached by the surgical tool, thereby providing means for the surgeon to avoid breaching out the pedicle's cortical bone. Accordingly, such electronic delays can be stored as control parameters 310 for use by the control module 305 and/or processor 307 for controlling the operation, selection and timing of one or more transducer elements 302 as defined by the control parameters 310.

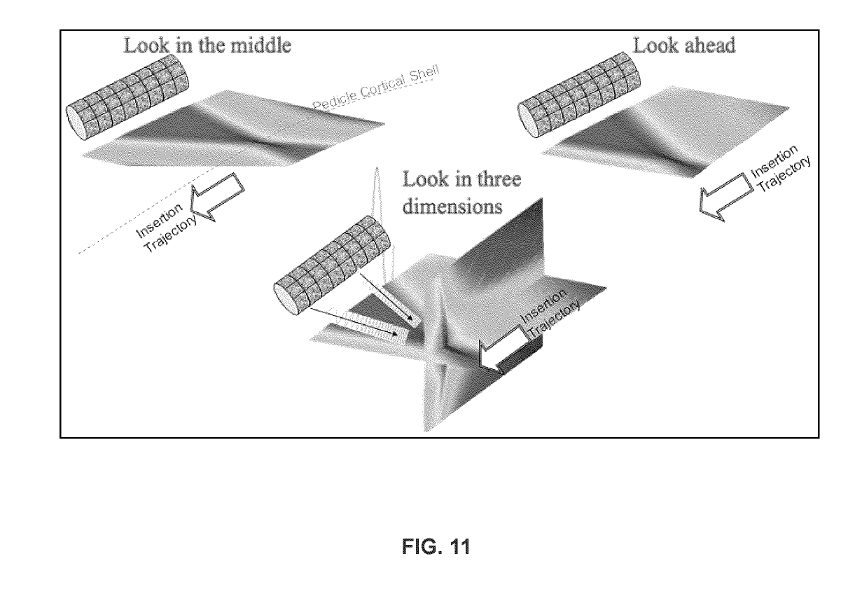

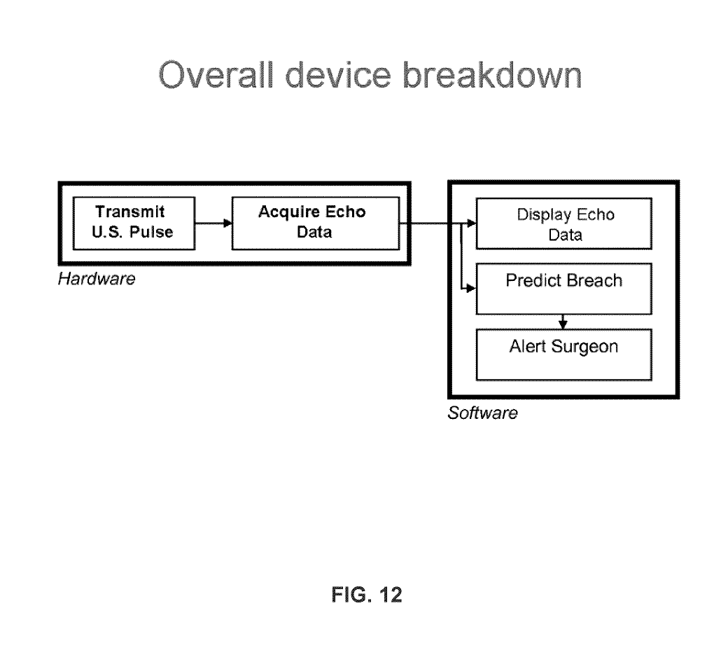

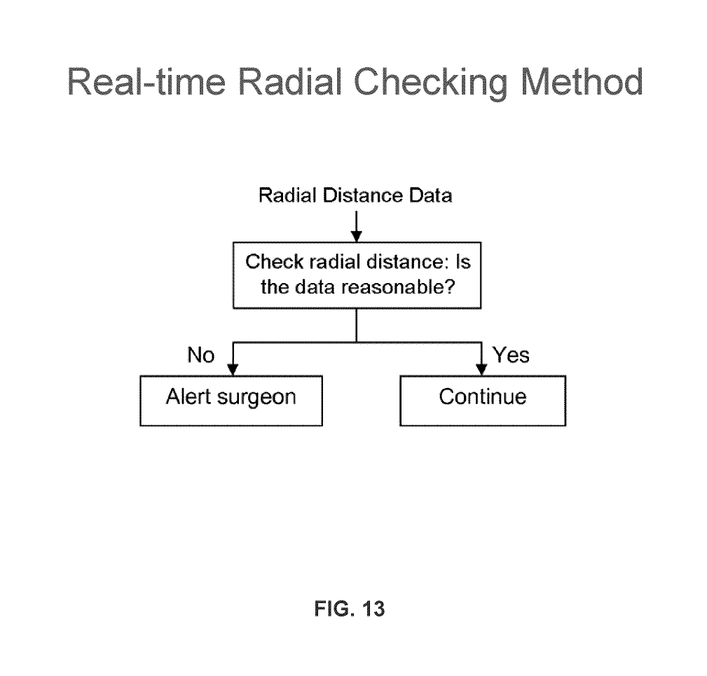

In some aspects, a method for predicting pedicle cortical breach is provided (FIG. 12). In some embodiments the method comprises inserting into the pedicle a tool comprising an annular ultrasound transducer, such as, for example, the annular transducer set forth in Example 2. In some embodiments of the method, the annular transducer is in communication with a computer processor (e.g. processor 316 of the computing device 302 in FIG. 3). Together, the transducer 301 and computer processor 316 are used to acquire the data required to produce a conical image of a pedicle bone, as described above. It is contemplated that the conical image acquired would include information regarding the location of the cortical boundary of the pedicle and relative to the inserted tool, which carries the annular array. It is contemplated that such an image could be used to predict the possibility for cortical breach based on the current insertional trajectory of the tool and the spatial relationship (e.g. radial distance between the tool and the cortical layer) of the tool relative to the cortical boundary. This information can be used to address the question “is the radial distance safe to continue drilling in the same trajectory (FIG. 13). If so, the user of the device (e.g. surgeon) continues drilling, unalerted. However, if the distance is deemed unsafe, then the processor (e.g. processor 307 in communication with processor 316) alerts the surgeon, reporting the estimated distance to breach.

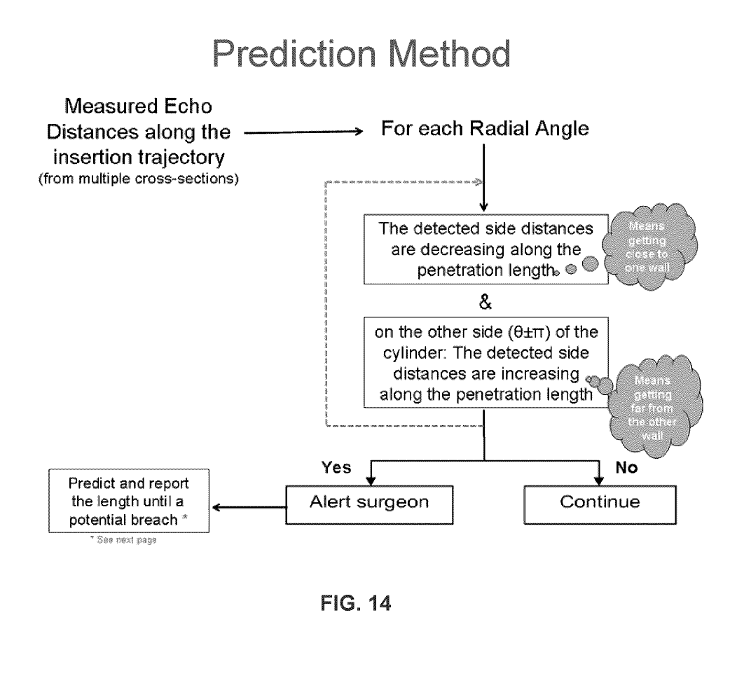

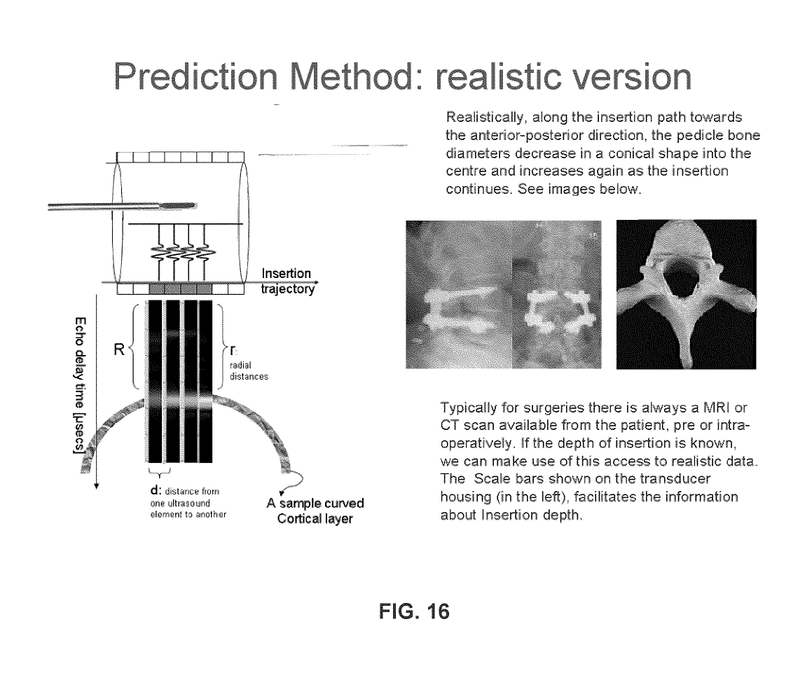

In some embodiments, a method for predicting cortical breach is provided. Referring to FIGS. 14, 15, and 16, the radial distance between the surgical tool and the cortical boundary of the pedicle is calculated. If the distance from the boundary is sufficient (e.g. at least 1-3 mm) the user is not alerted.

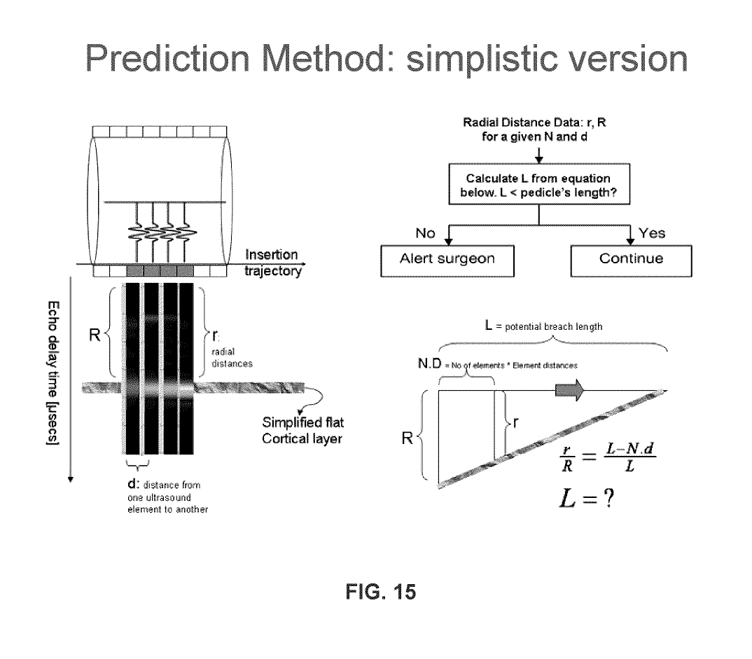

Implementation of the method referred to in FIGS. 14-16 involves use of the annular ultrasound transducer having a plurality of ring configurations, wherein transducer elements positioned on different rows are associated with specific angles that together form a line that coincides with a best-fit line (formed using the Least Squares Fitting method) that passes through the detected cortical walls at various cross-sections of a pedicle being probed. The length remaining prior to such a coincidence determines when a potential breach could occur at a given insertion trajectory (FIG. 15). This algorithm is particularly useful over short distances.

It is contemplated herein that in some embodiments of the present invention, the method can be used in conjunction with a medical image (e.g. MRI/CAT scan) of the targeted patient-specific pedicle anatomy to predict potential breaches of the pedicle. In some embodiments, a patient appearing in the operating room has with them at least one medical image such as CT or MRI of their spine. The method utilizes pre-operative and/or intra-operative three-dimensional images of a particular pedicle in conjunction with ultrasound imaging (e.g. as provided to the transducer array 301 from the computing device 302 and stored in database 317 or provided via user input 318) to guide drilling for the purposes of pedicle screw placement

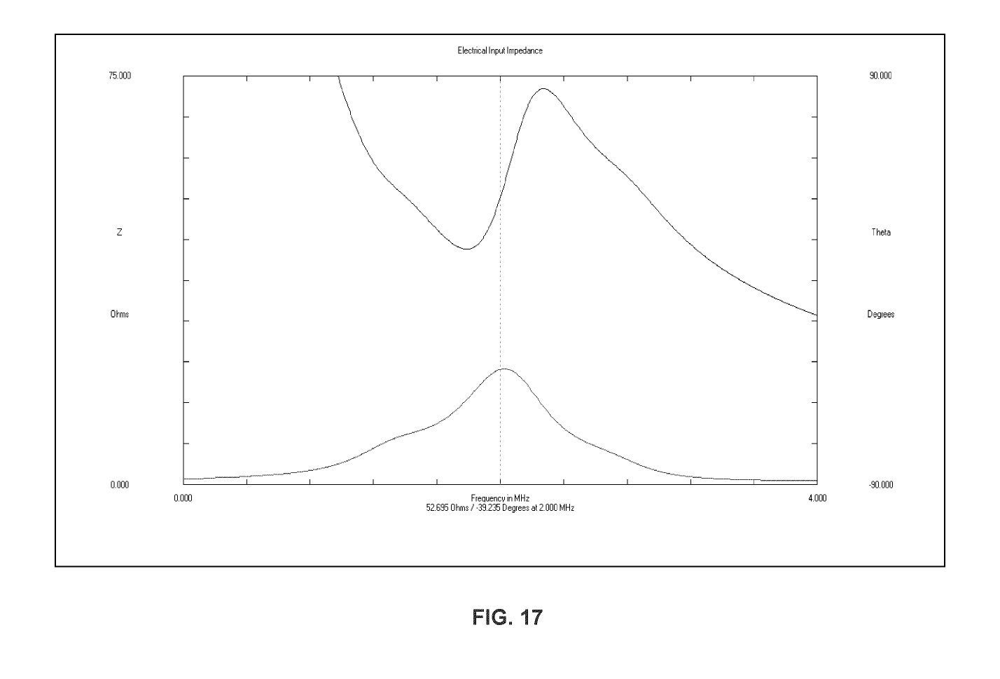

Referring to FIG. 17, if the depth of probe insertion is known (for example, by using scale bars on tool housing), software in communication with the annular ultrasound transducer having a plurality of ring configurations can be used to image various depth-specific cross-sections of the pedicle on a patient's scans and to provide an estimate of the curvature of the cortical wall of the pedicle being imaged. Reference to a patient's prior medical image of a pedicle (e.g. predefined parameters as stored on prior knowledge parameters 317 or provided via user interface 313) can be used as an alternative to the best-fit line algorithm described above. One advantage of using patient-specific scans is enhanced accuracy based on specific morphological information, particularly over predictions made of longer distances (e.g., >5 mm).

In order to achieve a better accuracy for potential cortical breach prediction, cross-sectional images can be overlapped on top of the pre- or intra-operative CT or MRI images of the specific target pedicle. The overlap, referred to herein as registration of multiple images together, illustrates how deep the drilling or insertion is relative to the patient's pedicle morphology. This is achieved by a scale bar on the transducer array device that shows the distance from the pedicle's guide hole opening relative to the closest circumferential sensors ring. Methods for image registration and overlap (also known as Computer Assisted Surgical Systems, CAS) are known in the art and can be applied to the images obtained using the ultrasound array, particularly when having access to patient scans. Examples for these techniques include, but are not limited to those described in U.S. Patent Publication No. 2011/0069867, U.S. Pat. No. 7,771,436 and algorithms used for Least-Squares fitting of two three-dimensional point clouds, such as the one named after “Arun”.

It is contemplated herein that embodiments of the methods are useful for screening bone tissue for evidence of osteoporosis. Ultrasound is safer, more portable and affordable than x-ray bone scans. In some embodiments, the ultrasound transducer of the present invention can be used by a medical practitioner to diagnose the health or disease state of bones that have limited access. For example, if a certain bone tissue has a known thickness but the ultrasound image generated using the transducer array of the present invention illustrates a longer distance than the known thickness, then the difference between the known thickness and measured distance would be indicative of a degree of osteoporosis. Indeed, a slower speed of ultrasound propagation would indicate less dense bone.

The invention will be more fully understood upon consideration of the following non-limiting Examples.

### EXAMPLES

### Example 1

### Cylindrical Transducer Array Configured in a Single Ring

Example 1 describes an annular array ultrasound transducer that is particularly useful for circumferential imaging of a single cross-section of bone tissue. The annular dimensions described herein, illustrate proof of principle, and are particularly useful for ultrasonic investigation of pedicle bones of the lumbar spine.

An exemplary low-frequency annular ultrasound transducer array was designed. Acoustic field simulation models were used to determine that an array having 32 elements with an aperture size of 4-8 elements (i.e., total number of active firing elements at a given time) provides a device having a good balance between image quality, practicality of use and cost-effective device fabrication. The annular rotational geometry of this design allows for improved focus of ultrasound waves and therefore improved image resolution and quality relative to a device having a single-element transducer (FIG. 6). FIG. 6 illustrates a graphical output of a schematic simulation depicting sound pressure profiles comparing the user of rotational phased arrays as compared to single element transducers. As illustrated in FIG. 6, it can be seen that the use of rotational phased arrays (as opposed to single element transducers) could lead to better focusing of the ultrasound waves, which results in sharper, clearer images as output from the image processor 316.

Device Electronics:

A combination of flex circuit and substrate with electrical path were used to wire individual elements to a coaxial cable. The following strategies were used for connecting the transducer with micro coaxial cables:

Flex circuit on the back and front layers for electrical connection, the flex circuit sample having a single 20 micron layer (e.g. other 20-80 μm layer may be envisaged). An electrical matching transformer with a 1:16 winding ratio was used. A coaxial cable bundle was used to wire the 32 elements and ground electrodes. Multiplexors were used to facilitate minimization of the circuitry.

Device Hardware Dimension and Material:

Referring to FIG. 2A, ¼ wavelength matching layers, (silver epoxy, Parylene), with 4 mm elevation; PZT thickness: 1.025 mm. Matching layer 1: silver epoxy: 0.16 mm; matching layer 2: parylene, LDPE: 0.27 mm; backing layer: epoxy loaded with tungsten/alumina: 1.57 mm, −6 db bandwidth (full width at half maximum): 69%.

Referring to FIG. 17, the cylindrical transducer array models described above had an electrical resistance of approximately 50Ω for each ultrasound element.

Electrical Impedance:

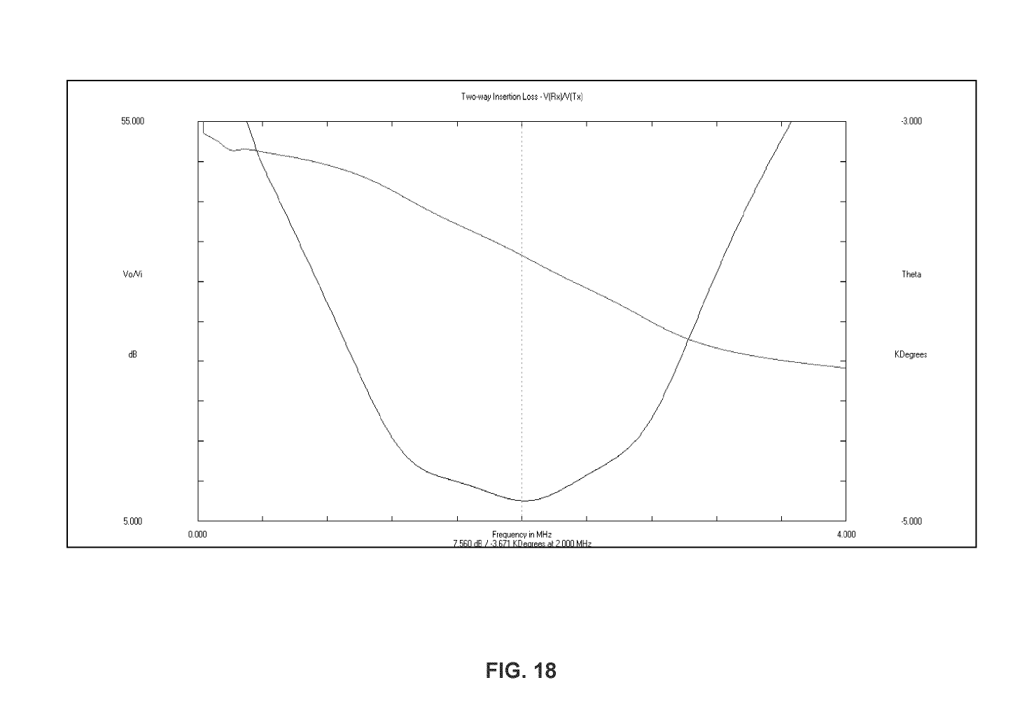

Referring to FIG. 18, electrical matching required for the low-frequency geometric array indicated around −8 dB (i.e., ˜40% ratio) energy loss at the interface of electronics with the acoustics hardware. An ideal transformer having a 1:16 ratio was applied.

Design Software:

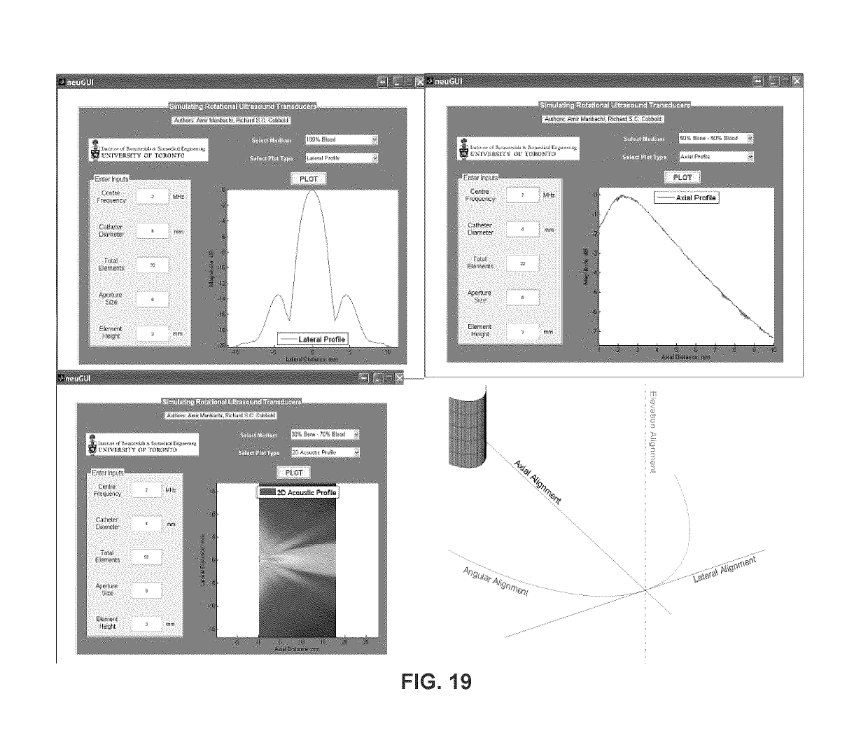

Referring to FIG. 19, a simulation modeling program based on the open-source software Field_II′ was created for designing the low-frequency rotational phased arrays disclosed herein. The design software and our graphical user interface generated for that are shown on FIG. 19. The simulation parameters and design characteristics were used to fabricate and setup the device consistent with operation of the application. The following exemplary input parameters were used to predict the behavior of the single ring transducer array described above:

1. center frequency of the design probe;

2. bandwidth (range of the frequencies) of the design probe;

3. dimensions of the probe, (height and total diameter);

4. total number of elements around the circumferential ring; and

5. aperture size (total number of elements active at a given point in time).

The software program generated plots of the sound pressure field in various settings separately (e.g., 2D acoustic profile, axial, lateral and elevation profiles), using the input parameters to generate a numerical value for image resolution in different directions.

### Example 2 (Prophetic)

### Annular Transducer Array Configured in Multiple Rings

Example 2 is a prophetic description of an annular ultrasound transducer that is particularly useful for generating multiple cross-sectional images of pedicle bone.

Typically, a piezoelectric material is chosen from a broad range of chemical compounds, the most common two materials being PZT (lead zirconate titanate) or PVDF (polyvinylidene fluoride). PZT provides a better sensitivity for imaging; however PVDF provides a wide-band performance in the frequency spectrum and is also more flexible for fabricating non-routine geometries. As an example for designing the thickness, PVDF is considered further here. PVDF has an associated speed of sound of 1500 [m/s]. The thickness of the PVDF element for imaging at a center frequency of 2 MHz needs to be at least: λ/2=c/2f=2250 [m/s]/(2*2 [MHz])=562.5 [μm] For fabrication purposes, it is reasonable to round up to a practical value of 750 [μm]. FIG. 7 depicts an exemplary cylindrical transducer array having multiple ring configurations as set forth in the present invention.

### Example 3 (Prophetic)

### Cortical Breach Prediction Using a Cylindrical Transducer Array Configured in Multiple Rings

Example 3 is a prophetic description of the cortical breach prediction, a method disclosed herein. In this method, ultrasound signals are radially transmitted and echoes received, as described above. As commonly implemented in the field, the amplitude of the echoes are converted into grayscale color map in order to arrive at the ultrasound brightness mode (B-mode) images for multiple cross-sectional images of the target bone.

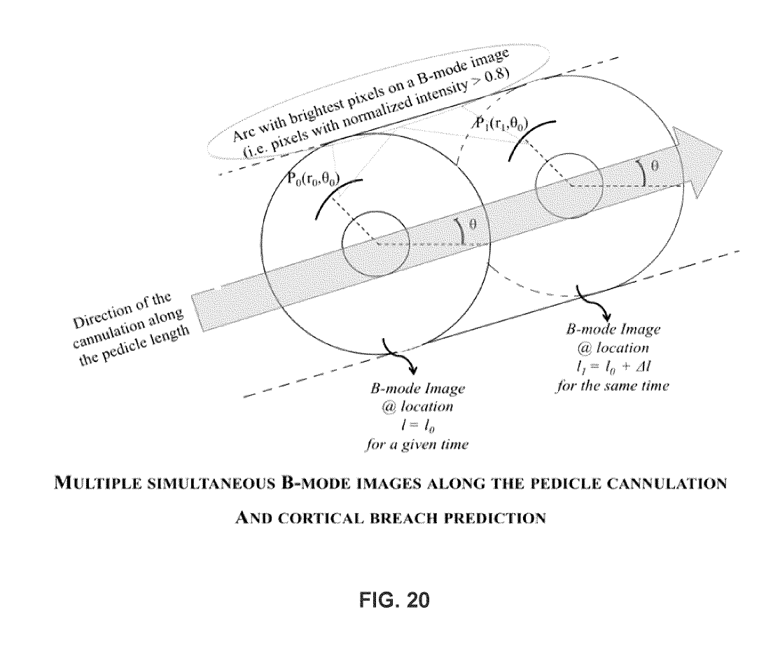

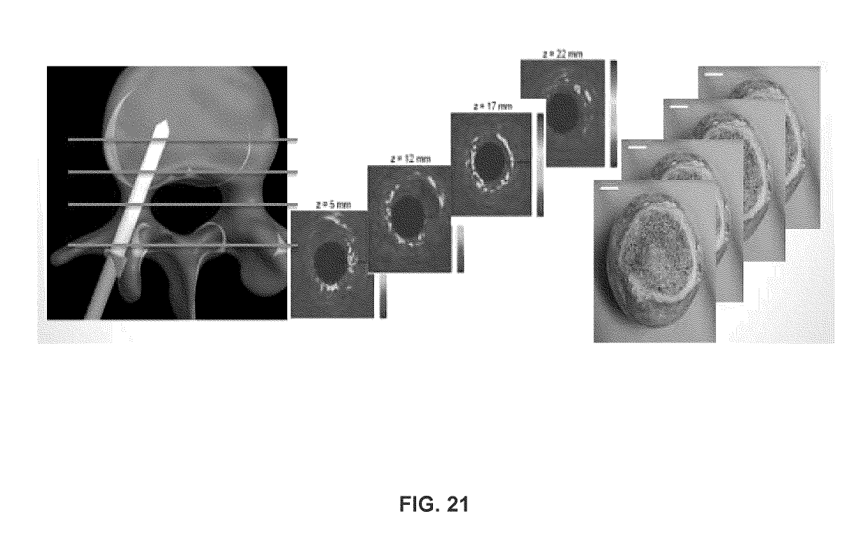

Referring to FIG. 20, the brightest pixels on a B-mode image (i.e. pixels with normalized intensity >0.8) are chosen as an arc (or few arcs). The arc is traced over the cannulation length for multiple cross sections at a specific time. For any fixed angle corresponding to middle of the arc (shown as angle θ in FIG. 5), if the radii of the images (r) follow the pattern of “r4<r3<r2<r1<r0” at θ and “r4>r3>r2>r1>r0” at 0+180°, then the trajectory chosen by the user is incorrect and will cause a pedicle breach if insertion is continued. Multiple cross sectional images are shown in FIG. 21.

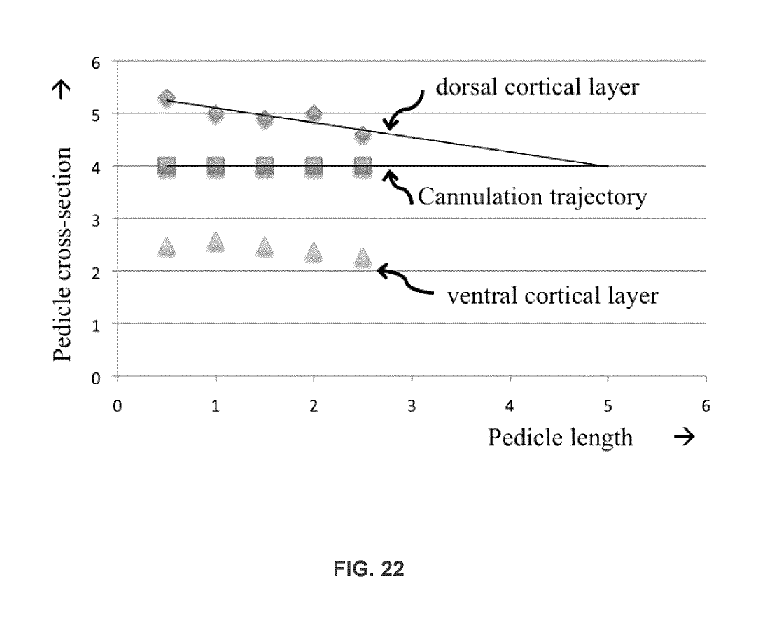

Based on the concept depicted in its simplest form on FIG. 22, the distance until potential cortical breach is calculated. If this distance is within the pedicle length, the user is alerted. Referring to FIG. 22, there is depicted a cannulation trajectory through a pedicle (marked in square) relative to the dorsal (marked in diamond) and vertical (marked in triangle) cortical layers. The trajectory shown will breach the dorsal cortical layer if its direction is not changed.

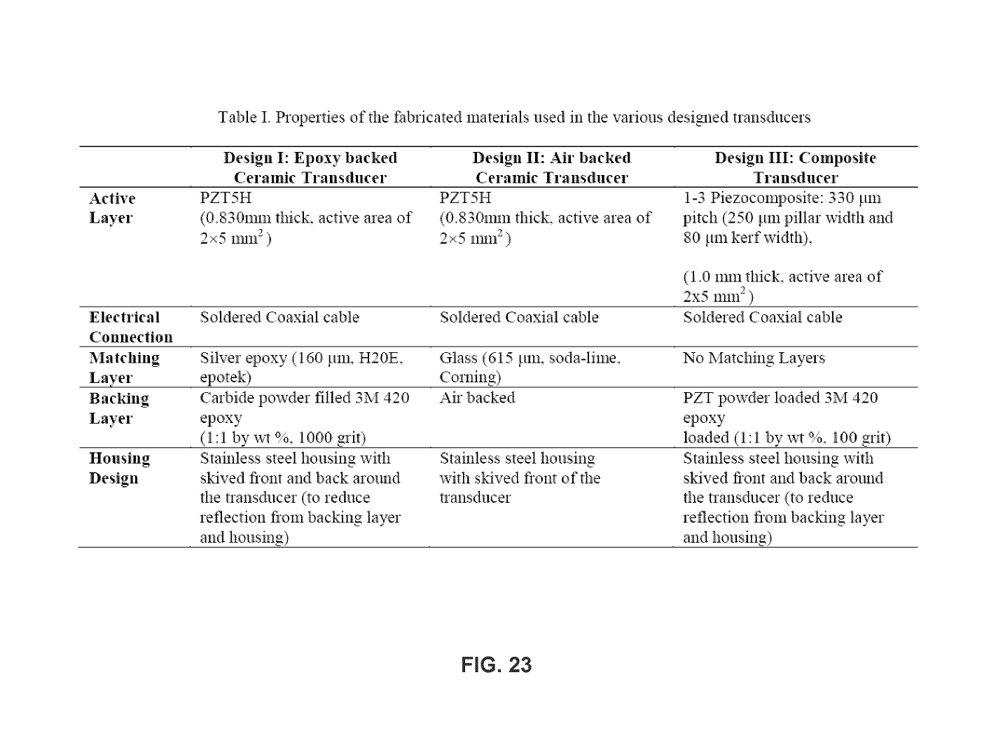

Referring to FIG. 23, shown are the properties of three different transducers, custom-designed and fabricated for bone sonography. The table summarizes the dimensions, as well as the types of fabricated materials used in the various designed transducers. The exemplary designs include: particle-loaded epoxy backed ceramic transducer, an air backed ceramic transducer, and a composite transducer (i.e. a mixture of ceramic based and polymer based transducers). In some embodiments, the composite structure may be preferable for bone sonography, at least because, where needed, by eliminating lateral and diagonal oscillation modes and introducing improved acoustic impedance matching, better sensitivity can be achieved relative to a ceramic based transducer. The epoxy- and air-backed ceramic designs are useful, for example, where wider bandwidth and higher resolution is desired. Parameters can be adjusted based on the user's preference and subjects anatomy and/or conditions such as osteoporosis.

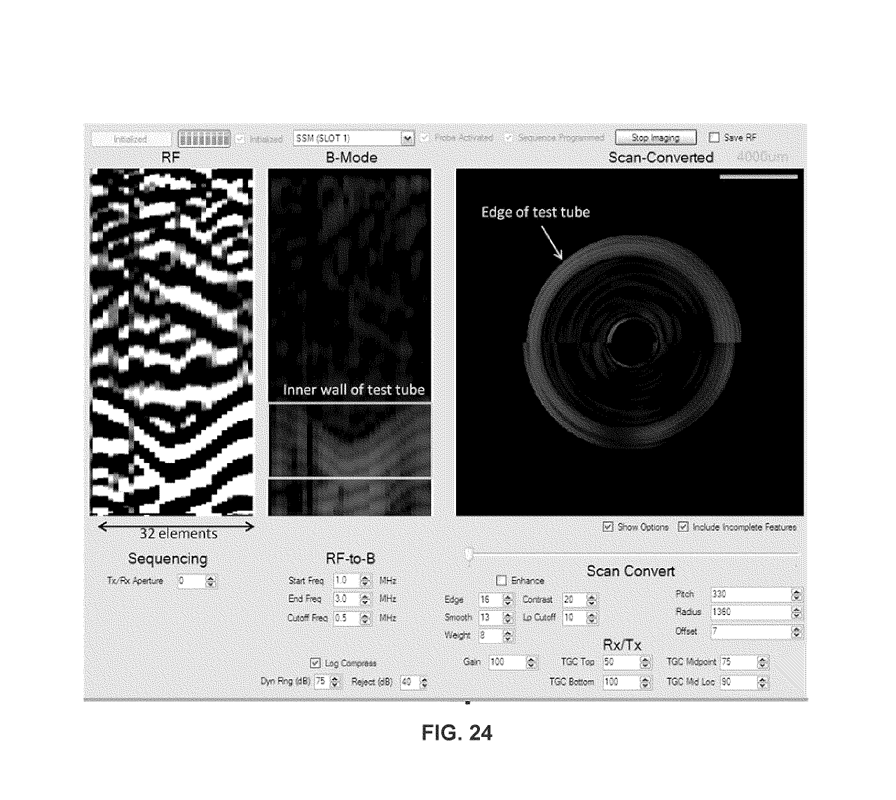

Referring to FIGS. 24-28 shown are exemplary screen views of a graphical user interface for a custom-developed C++/C# software that controls all the imaging driving parameters and depicts the captured images and raw data from the ultrasound transducer illustrated in FIG. 7.

A Demo interface allowing the user to initialize the hardware (please see top left corner on the screen), select various transducers connected to the ultrasound console through various slots (please see middle top feature, called SSM (SLOT 1) and start/stop imaging and save the Radio-frequency raw data as required. Is provided (see top right corner on the screen),

A two-dimensional linear map of the RF data (Radio-Frequency raw data) is provided on the left side of the screen. In this image, it is assumed that the 32 elements are sitting on the top and that each vertical line is a one-dimensional pulse-echo RF data.

A two-dimensional linear map of the B-mode data (brightness mode data) in the middle of the screen. In this image, it is assumed that the 32 elements are sitting on the top and that each vertical line is a one-dimensional pulse-echo RF data, being envelope detected, using available filters (e.g. Hilbert transform). The patterns seen on the top of this linear B-mode image are indicative of transmission signal combined with noise and speckle and are typically substantially static. The patterns seen towards the bottom half (depending on the distance of the imaging target echo) of this linear B-mode image are indicative of the echoes due to boundaries of the hollow structure of imaging interest. Such echo patters are typically motion-dependent and therefore vary in response to slight movement.

A Scan-Converted image (actual radial images from within the hollow structure being imaged) is provided on the right side of the screen. In this image, it is assumed that the 32-element phased array (e.g. component 301, in combination with the computing device 302 for providing the images via the display 315) is sitting in the middle of the Scan-converted image (the black circle in the center) whereby each of the vertical lines on the linear B-mode image constitutes radial data on a single angle, and the data associated with the angles in between two radial data are obtained based on a mathematical technique typically used in ultrasound systems, known as interpolation. The interface can provide a scale bar for reference purposes.

A “cine” feature enabling the obtained images to be played back in a cinematic loop is provided (see image sections with the scroll icon below the Scan-Converted image).

Referring further to FIGS. 24-28 shown are exemplary screen views of a graphical user interface of the custom-developed software that allows the user to employ various number of transducer array elements in imaging (see the section named “Sequencing” on the bottom left corner of the graphical user interface) For example, the user can select to use four, eight or one element at a time for imaging.

Referring further to FIGS. 24-28 shown are exemplary screen views of a graphical user interface of the custom-developed software that allows the user to use the section “RF-to-B” (Radio-Frequency to Brightness mode ultrasound image) in order to adjust the frequency spectrum of the transmission signal and dynamic range of the image. (see the section named “RF-to-B” on the bottom of the graphical user interface)

Referring further to FIGS. 24-28 shown are exemplary screen views of a graphical user interface of the custom-developed software that allows the user to vary geometric settings (such as the diameter of the array, the element spacing, pitch, etc.), and the post-processing features common to many ultrasound imaging system, such as Time-Gain Compensation and Speckle Reduction techniques. (see the section named “Scan Convert” on the bottom right corner of the graphical user interface)

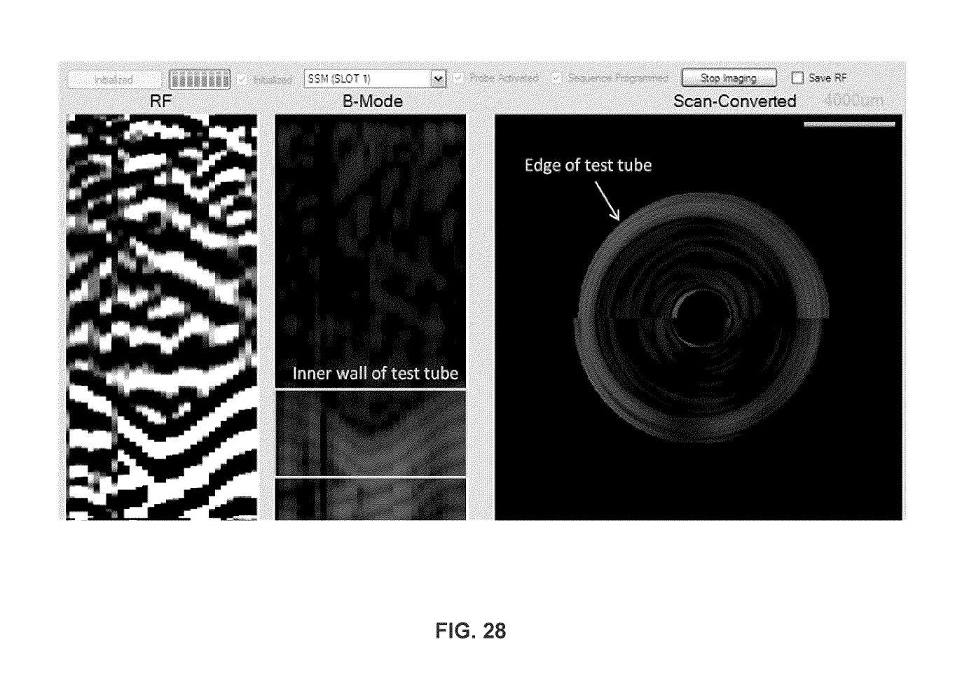

Referring to FIG. 28 shown are exemplary screen views of a graphical user interface of the custom-developed software that allows the user to disable all the technical options and controlling or processing features, in order to just see the clinically relevant images.

The transducer array was tested on the following experiments: 1. Detecting the edges of a glass test tube filled with water surrounding the array; 2. Detecting the cortical layer of human pedicle bone samples.

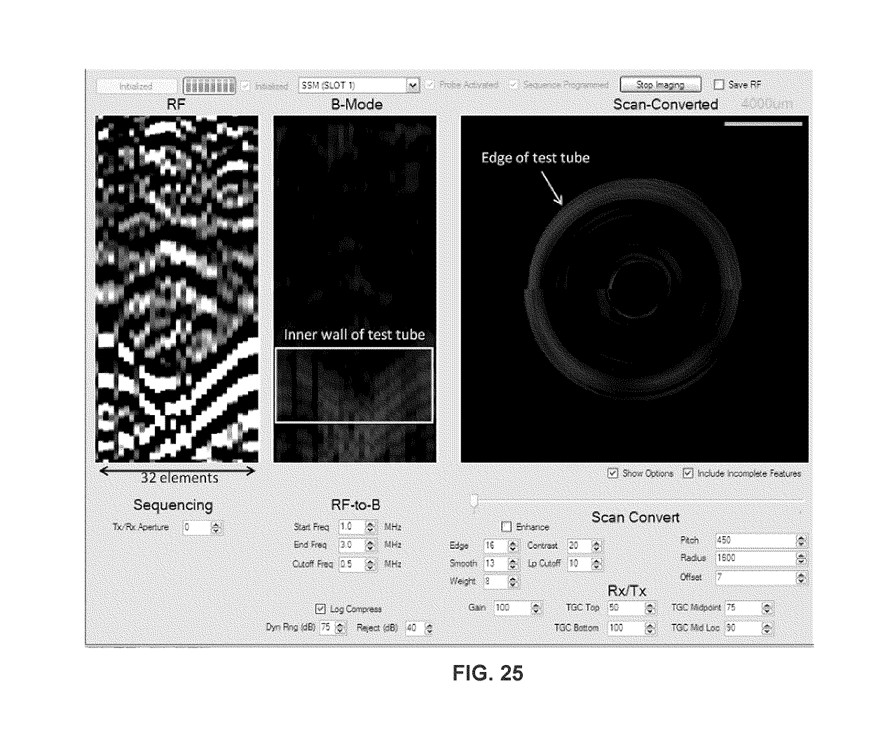

Referring to FIGS. 24-25, shown are exemplary screen views of a graphical user interface of the custom-developed software when used to test the capability of the array 301 in detecting the edges of a glass test tube filled with water surrounding the array. The array 301 and the glass boundaries of the hollow structure are labeled both on the Scan-converted and linear B-mode images.

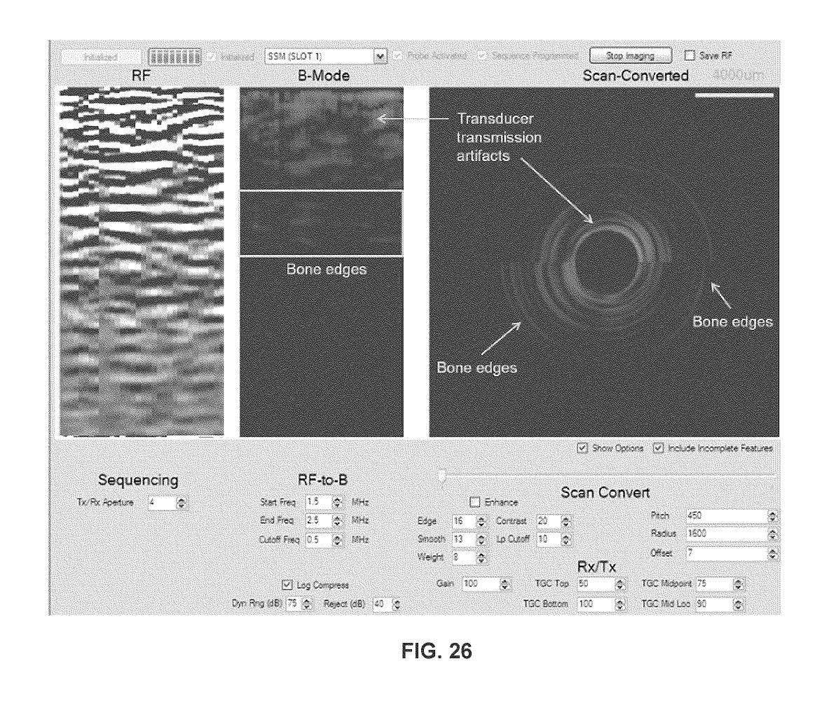

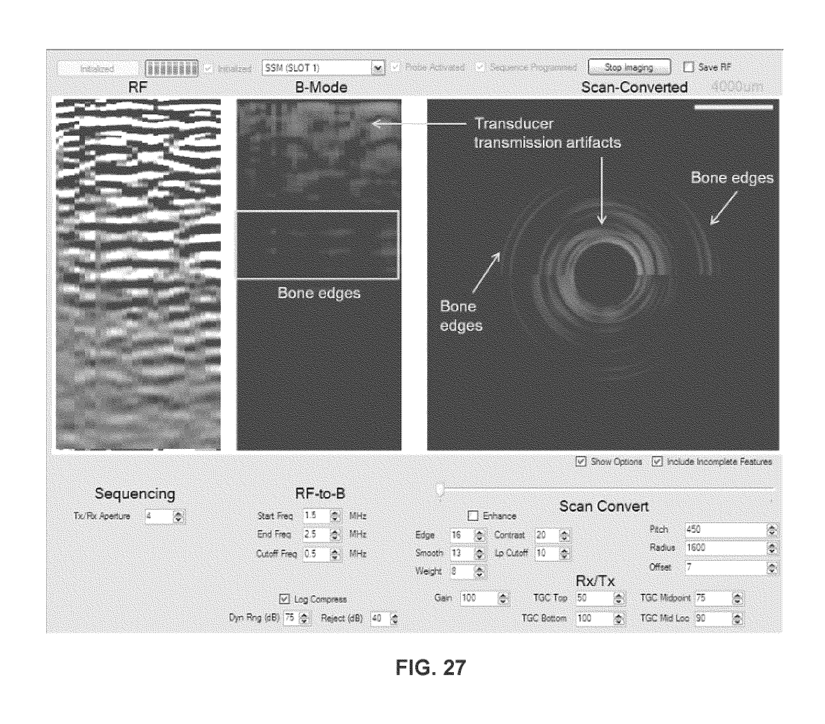

Referring to FIGS. 26-27, shown are exemplary screen views of a graphical user interface of the custom-developed software when used to test the capability of the array 301 in detecting the cortical layer of a human pedicle bone sample (thoracic, T10 level). The array 301 and the cortical layer boundaries of the pedicle's structure are labeled both on the Scan-converted and linear B-mode images.

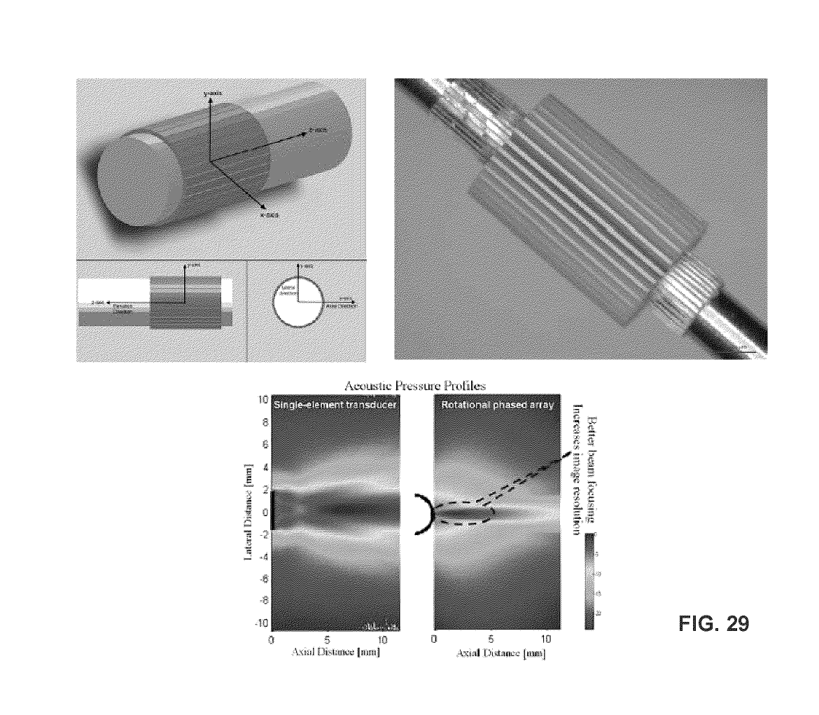

Referring to FIG. 29, shown is an example of a cylindrical phase array ultrasound transducer. Top images show the array that eliminates the need for mechanical rotation and which generates radial images from within the pedicle bore hole. Bottom image illustrates computer simulations of acoustic pressure profiles that suggest that the use of a phased cylindrical array (e.g. 301) in combination with the computing device 302 for providing the images via the display 315 (as shown in FIG. 3) could be advantageous in some embodiments, as it could provide improved focusing and higher resolution images.

Although the invention has been described with reference to certain specific embodiments, various modifications thereof will be apparent to those skilled in the art without departing from the purpose and scope of the invention as outlined in the claims appended hereto.

Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

1. An annular ultrasound transducer array comprising:
a plurality of transducer elements arranged in a ring configuration, wherein the plurality of transducer elements comprises elements configured to transmit ultrasound signals and elements configured to received ultrasound echoes, and wherein the ultrasound signal is transmitted at a frequency in a range of 0.5 to 5 MHz.

2. The annular ultrasound transducer array of claim 1, further comprising a plurality of the ring configurations, wherein the plurality of ring configurations are arranged adjacent to one another in a cylindrical configuration, each ring forming a row in the cylindrical configuration.

3. The annular ultrasound transducer array of claim 1 or 2, wherein the transducer array is phased.

4. The annular ultrasound transducer array of claim 2 or 3, wherein the transducer elements in every other row are transmitters and the transducer elements in the rows between the transmitters are receivers.

5. The annular ultrasound transducer array of any one of claims 1 to 4, wherein the diameter of the ring configuration is in a range of 3 to 5 mm.

6. The annular ultrasound transducer array of any one of claims 1 to 5, wherein the transducer array is configured to be mounted on or in a tool for probing or cannulating bone.

7. The annular ultrasound transducer array of any one of claims 1 to 6, wherein the transducer array is integrated with a tool for probing or cannulating bone.

8. The annular ultrasound transducer array of claim 6 or 7, wherein the tool is for generating pedicle guide holes or pedicle screw placement.

9. The annular ultrasound transducer array of any one of claims 1 to 8, wherein the ultrasound signal to be transmitted is processed by coded excitation and wherein the received ultrasound echoes are processed by de-coding of coded excitation.

10. The annular ultrasound transducer array of any one of claims 1 to 9, wherein the plurality of transducer elements are configured to transmit ultrasound signals in a non-simultaneous, sequential manner.

11. The annular ultrasound transducer array of any one of claims 2 to 10, wherein the transmitted ultrasound signals are directionally focused at an angle less than 90 degrees relative to the longitudinal axis of the cylindrical configuration.

12. The annular ultrasound transducer array of any one of claims 1 to 11, wherein the transducer is in communication with an imaging processor.

13. The annular ultrasound transducer array of any one of claims 1 to 12, wherein the image is generated in real time.

14. A method for producing an image using an ultrasound system, the method comprising:
a) acquiring ultrasound data by:
i) transmitting a plurality of ultrasound signals directed outwardly at a bone to be imaged, wherein the signals are transmitted at frequencies in the range of 0.5 to 5 MHz, wherein the signals are reflected by features within the imaged object to produce echoes;
ii) measuring the echoes, wherein the measured echoes include echoes reflected from multiple spatial locations within the bone to be imaged;
b) producing an image of the bone from the received echoes.

15. The method of claim 14, wherein the outwardly directed ultrasound signals are transmitted by a plurality of transducer elements arranged in a ring configuration, wherein the echoes are received by the plurality of transducer elements, wherein the plurality of transducer elements are in communication with an imaging processor, and wherein the image produced is a cross-sectional image.

16. The method of claim 14, wherein the outwardly directed ultrasound signals are transmitted by a plurality of transducer elements arranged in a first plurality of ring configurations, wherein the echoes are received by a second plurality of ring configurations, wherein the first and second plurality of ring configurations are arranged adjacent to one another in a cylindrical configuration, each ring forming a row in the cylindrical configuration and wherein the image produced is a cylindrical or conical image.

17. The method of claim 16, wherein the plurality of adjacent rings are mounted to or in or integrated with a tool and wherein the tool is inserted in the object to be imaged.

18. The method of claim 17, further comprising ultrasound signals directed forwardly relative to the insertional trajectory of the tool, wherein the forwardly directed ultrasound signals are transmitted from a plurality of the transducer elements and wherein the image produced is a conical image, wherein the apex of the cone is ahead of the tool along the insertional axis.

19. The method of any one of claims 14 to 18, wherein the imaged bone is a pedicle bone.

20. The method of any one of claims 14 to 19, wherein the image is generated in real time.

21. A method for predicting pedicle cortical breach, the method comprising:
a) inserting into the pedicle a tool comprising an annular ultrasound transducer;
b) acquiring ultrasound data by:
i) transmitting from the annular ultrasound transducer a plurality of ultrasound signals directed both outwardly and forwardly relative to the insertional trajectory of the tool, wherein the signals are transmitted at a frequency in a range of 0.5 to 5 MHz, wherein the signals are reflected by features within the pedicle to produce echoes;
ii) measuring the echoes using a the annular ultrasound transducer, wherein the measured echoes include echoes reflected from multiple spatial locations within the pedicle;
c) producing an image of the pedicle from the received echoes, wherein the image includes the cortical boundary of the pedicle, wherein a spatial relationship between the inserted tool and the cortical boundary is depicted in the image
d) predicting the possibility for cortical breach based on the image obtained in step c).

22. The method of claim 21, wherein the tool is a cannulation probe or drill.

23. The method of claim 21 or 22, wherein the ultrasound signals to be transmitted are processed by coded excitation and wherein the echoes are processed by de-coding.

24. A system for producing an image of bone using an ultrasound system, the system comprising:
a) a phased annular transducer comprising a plurality of transducer elements arranged in a ring configuration, wherein the plurality of transducer elements comprises elements configured to transmit ultrasound signals and elements configured to received ultrasound echoes, and wherein the ultrasound signal is transmitted at a frequency in a range of 0.5 to 5 MHz;
b) a tool configured to probe or cannulate bone, wherein the tool comprises the phased annular transducer;
c) an imaging processor in communication with the phased annular transducer;
d) an imaging display coupled with the imaging processor; and
e) an electronic controller coupled with the tool and the phased annular transducer, wherein the electronic controller is configured to control the operation of the tool to move the tool in a desired direction.

25. The system of claim 24, wherein the phased annular transducer further comprises a plurality of the ring configurations, wherein the plurality of ring configurations are arranged adjacent to one another in a cylindrical configuration, each ring forming a row in the cylindrical configuration.

26. The system of claim 24, further comprising:
a processor in communication with the array, the processor comprising a memory and instructions stored thereon, the instructions when executed for generating control signals to control transmitting the modulated ultrasound signal in accordance with pre-defined parameters.

27. The system according to claim 26, wherein the parameters comprise: a selection of one or more transducer elements for transmitting a modulated ultrasound signal from the ultrasound transducer array, a directional angle of the transmitted ultrasound signal, a focus direction of the ultrasound transducer array, and time delays for transmitting the ultrasound signal.

28. The method according to claim 1, further comprising: generating the image on a display in dependence upon the representative echo signal.
